SEQUENCE LISTING (64 sequences)

SEQ ID NO: 1 - protein, 8 aa, Artificial Sequence
  CDRH3 sequence
ARDYGLDQ

SEQ ID NO: 2 - protein, 12 aa, Artificial Sequence
  CDRH3 sequence
VKDPKAWLEPEW

SEQ ID NO: 3 - protein, 12 aa, Artificial Sequence
  CDRH3 sequence
ARDNRAVDGFDI

SEQ ID NO: 4 - protein, 6 aa, Artificial Sequence
  CDRH3 sequence
ARDHVH

SEQ ID NO: 5 - protein, 9 aa, Artificial Sequence
  CDRH3 sequence
TRGDYVFVY

SEQ ID NO: 6 - protein, 14 aa, Artificial Sequence
  CDRH3 sequence
AREQRSTWLNGMDV

SEQ ID NO: 7 - protein, 15 aa, Artificial Sequence
  CDRH3 sequence
ARDRMDYSGSGVFDY

SEQ ID NO: 8 - protein, 12 aa, Artificial Sequence
  CDRH3 sequence
TFGIHWANPFDI

SEQ ID NO: 9 - protein, 15 aa, Artificial Sequence
  CDRH3 sequence
ARGTVNFYDNRCLDY

SEQ ID NO: 10 - protein, 8 aa, Artificial Sequence
  CDRH3 sequence
MTHYGLTY

SEQ ID NO: 11 - protein, 11 aa, Artificial Sequence
  CDRH3 sequence
ARDRAEQHFDY

SEQ ID NO: 12 - protein, 12 aa, Artificial Sequence
  CDRH3 sequence
AREVVEGYYMDV

SEQ ID NO: 13 - protein, 10 aa, Artificial Sequence
  CDRH3 sequence
ARVGGSYIGY

SEQ ID NO: 14 - protein, 9 aa, Artificial Sequence
  CDRH3 sequence
ARGDYVFVY

SEQ ID NO: 15 - protein, 16 aa, Artificial Sequence
  CDRH3 sequence
ARPKAVGFYHVGYFDL

SEQ ID NO: 16 - protein, 9 aa, Artificial Sequence
  CDRH3 sequence
ARESGYFDY

SEQ ID NO: 17 - protein, 11 aa, Artificial Sequence
  CDRL3 sequence
QVWDSDSYHYV

SEQ ID NO: 18 - protein, 9 aa, Artificial Sequence
  CDRL3 sequence
QQSYSNPRT

SEQ ID NO: 19 - protein, 9 aa, Artificial Sequence
  CDRL3 sequence
QQSYSNLFT

SEQ ID NO: 20 - protein, 9 aa, Artificial Sequence
  CDRL3 sequence
LYYNSAPWT

SEQ ID NO: 21 - protein, 9 aa, Artificial Sequence
  CDRL3 sequence
QQYGSSPWT

SEQ ID NO: 22 - protein, 9 aa, Artificial Sequence
  CDRL3 sequence
QHRSTWPPT

SEQ ID NO: 23 - protein, 9 aa, Artificial Sequence
  CDRL3 sequence
QQYGSYSRT

SEQ ID NO: 24 - protein, 10 aa, Artificial Sequence
  CDRL3 sequence
SSYAGSNDFE

SEQ ID NO: 25 - protein, 9 aa, Artificial Sequence
  CDRL3 sequence
QQSFSTPRT

SEQ ID NO: 26 - protein, 13 aa, Artificial Sequence
  CDRL3 sequence
GAEHGSGSNFLVV

SEQ ID NO: 27 - protein, 11 aa, Artificial Sequence
  CDRL3 sequence
HLRRNWPPALT

SEQ ID NO: 28 - protein, 11 aa, Artificial Sequence
  CDRL3 sequence
CSYVGSSTSYV

SEQ ID NO: 29 - protein, 10 aa, Artificial Sequence
  CDRL3 sequence
QQYGSSPPYT

SEQ ID NO: 30 - protein, 9 aa, Artificial Sequence
  CDRL3 sequence
QQYDTSPWT

SEQ ID NO: 31 - protein, 10 aa, Artificial Sequence
  CDRL3 sequence
QQYNNWPPGT

SEQ ID NO: 32 - protein, 10 aa, Artificial Sequence
  CDRL3 sequence
QQYNNWPTWT

SEQ ID NO: 33 - protein, 115 aa, Artificial Sequence
  Heavy chain variable region sequence
QVQLVQSGGGLVRPGGSLRLSCVASGFTFSSFEMNWVRQAPGKGLEWVAYIGDSGSTIFYADSVQGRFTISRDNAKASLYLQMNDLRAEDTAIYYCARDYGLDQWGQGTLVTVSS

SEQ ID NO: 34 - protein, 108 aa, Artificial Sequence
  Light chain variable region sequence
QPVLTQPPSVSVAPGQTASLTCAGHNIGDKSVHWYQQKPGQAPVEVIFHDSERPPGIPERFSASNSGNTAILTISRVEAGDEADYHCQVWDSDSYHYVFGGGTKLTVL

SEQ ID NO: 35 - protein, 118 aa, Artificial Sequence
  Heavy chain variable region sequence
QVQLVQSGGDLVQPGGSLRLSCAASGFTFSSYVMSWVRQAPGKGLEWVSVIYRGGSTKYADSVKGRFTISRDDSKNTLYLQMNSLRVEDTAVYYCVKDPKAWLEPEWWGQGTLVTVSS

SEQ ID NO: 36 - protein, 107 aa, Artificial Sequence
  Light chain variable region sequence
DIVLTQSPSSLSASVGDRVTITCRASQSISKYLSWFQQKPGKAPNLLIYAASYLQSGVPSRFSGSGSGTDFTLTISSLQPEDFATYYCQQSYSNPRTFGQGTKVEIK

SEQ ID NO: 37 - protein, 119 aa, Artificial Sequence
  Heavy chain variable region sequence
QVQLVQSGGGLVRPGGSLRLSCAASGFTFTTFRMSWVRQAPGKGLEWVANINQDSSEKYYVDSVKGRFSISRDNAKNSLYLQMNSLRAEDTAVYYCARDNRAVDGFDIWGQGTTVTVSS

SEQ ID NO: 38 - protein, 107 aa, Artificial Sequence
  Light chain variable region sequence
ETTLTQSPSSLSASVGDRVTITCRASQSIYSYLNWYQKKIGKAPKLLIYAASSLQSGVPSRFSGSGSGTDFTLTISSLQPEDIATYYCQQSYSNLFTFGPGTKVEIK

SEQ ID NO: 39 - protein, 113 aa, Artificial Sequence
  Heavy chain variable region sequence
QVQLVQSGGGLVKPGGSLRLSCAASGFSLSSYSMNWVRQAPGKGLEWVSSISNTGSYKYYADSVKGRFTISRDNAKNSVYLQMNSLRAEDRAVYYCARDHVHWGQGTLVTVSS

SEQ ID NO: 40 - protein, 77 aa, Artificial Sequence
  Light chain variable region sequence
ETTLTQSPSSLSASVGDRVTITCRASQGISNFLAWYQQKPGKVPKLLIYTTSTLQSGVPSRFSGSGSGTDFTLTISS

SEQ ID NO: 41 - protein, 118 aa, Artificial Sequence
  Heavy chain variable region sequence
EVQLVESGGGLVQPGRSLRLSCSASGFTFGDYGMTWVRQAPGKGLEWVGLVRSEAYRGTTEYAASVRGRFTISRDNARNIAYLHMNSLKTEDTGVYYCTRGDYVFVYWGQGTLVTVSS

SEQ ID NO: 42 - protein, 108 aa, Artificial Sequence
  Light chain variable region sequence
ETTLTQSPGTLSLSPGERATLSCRASQSVSNNYLAWYQQKPGQAPRFLIYRASSRATGIPDRFSGTGSGTDFTLTISRLEPEDFAVYYCQQYGSSPWTFGQGTKVDIK

SEQ ID NO: 43 - protein, 121 aa, Artificial Sequence
  Heavy chain variable region sequence
QITLKESGAEVKKPGASVKVSCKASGYTIGSYGISWVRQAPGQGLEWMGWISGNNDNTNYVEKFQGRVIMTIDTSTSTAYMELRSLTSDDTAVYYCAREQRSTWLNGMDVWGQGTTVTVSS

SEQ ID NO: 44 - protein, 107 aa, Artificial Sequence
  Light chain variable region sequence
EIVMTQSPATLSLSPGERATLSCRASQSVSRYLAWYQQKPGQAPRLLIYDSSNRATGVPARFSGSGSGTDFTLTISSLEPEDFAVYYCQHRSTWPPTFGPGTKVEIK

SEQ ID NO: 45 - protein, 123 aa, Artificial Sequence
  Heavy chain variable region sequence
QVQLVESGPGLLKPSQTLSLTCTVSGGSLSSGGYYWSWIRQHPGQGLECIGYIYYSGSTYYSPSLESRVDISMDTSMNQFSLKLRSVTAADTAVYYCARDRMDYSGSGVFDYWGQGTLVTVSS

SEQ ID NO: 46 - protein, 107 aa, Artificial Sequence
  Light chain variable region sequence
EIVLTQSPSTLSASVGDRVTITCRASQSISRWLAWYQQKPGKAPRLLIHKASSLESGVPSRFSGSGSGTEFTLTITSLQPDDFATYYCQQYGSYSRTFGQGTKVEIK

SEQ ID NO: 47 - protein, 119 aa, Artificial Sequence
  Heavy chain variable region sequence
QVQLVQSGAEVKKPGASVNVSCKASGYTFPSYGISWVRQAPGQGLEWMGWISPYSGNTNYAQKLQGRVIMTTDPSTSTAYMDLRSLTSDDTAVYYCTFGIHWANPFDIWSQGTTVTVSS

SEQ ID NO: 48 - protein, 110 aa, Artificial Sequence
  Light chain variable region sequence
QPVLTQPPSASGSPGQSVTISCTGTSSDVGGYNYVSWYQQHPGKAPKLMIYEVSKRPSGVPDRFSGSKSGNTASLTVSGLQAEDEADYYCSSYAGSNDFEFGGGTKLTVL

SEQ ID NO: 49 - protein, 121 aa, Artificial Sequence
  Heavy chain variable region sequence
QVQLQQWGAGLLKPSETLSLTCAVYGGSFSGYYWSWIRQSPGKGLEWIGEINHSGTTHYNPSLNSRVTMSVDTSKSQFSLNLSSVTAADTAVYYCARGTVNFYDNRCLDYWGQGTLVTVSS

SEQ ID NO: 50 - protein, 107 aa, Artificial Sequence
  Light chain variable region sequence
DIQMTQSPSSLSASVGDRVIITCRASHSISSYLNWYQQKAGKAPKLLIYAASSLQSGVPSRFSGSGSGTDFSLTISSLQPEDFATYYCQQSFSTPRTFGQGTKVDIK

SEQ ID NO: 51 - protein, 117 aa, Artificial Sequence
  Heavy chain variable region sequence
EVQLVESGGGLVKPGGSLRLSCAASGFTFSHGWMSWVRQAPGKGLEWVGRIKRKTDAGTIDYAAAVKGRFTISRDDSKNTLYLQMNSLKMEDTAVYYCMTHYGLTYWGQGTLVTVSS

SEQ ID NO: 52 - protein, 116 aa, Artificial Sequence
  Light chain variable region sequence
QPVLTQPPSASASLGASVTLTCTLSSDYSTYKVDWYQQRPAKGPRFVMRVGTGGIVGSKGDGIPDRFSALGSGLNRYLIIKNIQQEDEGDYHCGAEHGSGSNFLVVFGGGTKLTVL

SEQ ID NO: 53 - protein, 119 aa, Artificial Sequence
  Heavy chain variable region sequence
QVQLVQSGAEVKKPGASVKVSCKASGYSFTGYSMHWVRQAPGQGLEWMGWISPSSGVANYAQKFQGRVTMTTDTSITTAYMELSRLRSDDTAVYYCARDERAEQHFDYWGQGTLVTVSS

SEQ ID NO: 54 - protein, 109 aa, Artificial Sequence
  Light chain variable region sequence
EIVMTQSPATLSLSPGERATLSCRASQSLSSYLAWYQQKPGQAPRLLIYDTSNRATGIPARFSGSGSGTDFTLTISSLEPEDFAVYYCHLRRNWPPALTFGGGTKVEIK

SEQ ID NO: 55 - protein, 119 aa, Artificial Sequence
  Heavy chain variable region sequence
QVQLVESGPGLVKPSETLSLTCAVSGYSISIGYFWGWIRQPPGKGLEWIGSIYHGGSTYYNPSLKSRVTMSVDTSKNQFSLKLRSVTAADTAIYYCAREVVEGYYMDVWGKGTTVTVSS

SEQ ID NO: 56 - protein, 111 aa, Artificial Sequence
  Light chain variable region sequence
QSALTQPASVSGSPGQSITISCTGTSSHVGNYNLVSWYQHHPGKAPKLVISEVNKRPSGISNRFSGSKSGNTASLTISGLQAEDEADYYCCSYVGSSTSYVFGGGTKLTVL

SEQ ID NO: 57 - protein, 117 aa, Artificial Sequence
  Heavy chain variable region sequence
QVQLVQSGPEVKKPGASVKVSCKASGYTFSTYGIIWVRQAPGQGPECIGWISAHNGNTKYAQNLQGRLTLTTDTSTSTAYMELRSLRSDDTAVYYCARVGGSYIGYWGQGTLVTVSS

SEQ ID NO: 58 - protein, 109 aa, Artificial Sequence
  Light chain variable region sequence
EIVLTQSPGTLSLSPGERATLSCRASQSVTNSYLAWYQQKPGQAPRLLIYGASSRATGIPDRFSGSGSGTDFTLTISRLEPEDFAVYYCQQYGSSPPYTFGQGTKVEIK

SEQ ID NO: 59 - protein, 118 aa, Artificial Sequence
  Heavy chain variable region sequence
EVQLVESGGGLVQPGRSLRLSCTASGFTFGDYAMSWVRQAPGKGLEWVGFIRSEAYGGATEYAASVKGRFTISRDNSKSIAYLQMNSLRTEDTALYYCARGDYVFVYWGQGALVTVSS

SEQ ID NO: 60 - protein, 108 aa, Artificial Sequence
  Light chain variable region sequence
EIVMTQSPGTLSLSLGERATLSCRASQSVSSNYLAWYQQKPGQAPELLIYRASSRATGIPDRFSGSGSGTDFTLTISTLEPEDFAVYYCQQYDTSPWTFGQGTKVEIK

SEQ ID NO: 61 - protein, 124 aa, Artificial Sequence
  Heavy chain variable region sequence
QVQLVESGPGLVKPSETLSLTCTVSGGSISSSSLFWGWIRQSPGKGPEWIGSIYDSVNTYYNPSLQSRVTISVDTSKNQFSLNLRSVTVADTAVYYCARPKAVGFYHVGYFDLWGRGTTVTVSS

SEQ ID NO: 62 - protein, 108 aa, Artificial Sequence
  Light chain variable region sequence
DIVMTQTPATLSVSPGERATLSCRASQSVSSSLAWYQQKTGQAPRLLIYGASTRATGIPARFSGSGSGTEFTLTISSLQSEDFAVYYCQQYNNWPPGTFGQGTKVEIK

SEQ ID NO: 63 - protein, 117 aa, Artificial Sequence
  Heavy chain variable region sequence
EVQLLESGPGLVKPSETLSLTCTVSGGLISSSSYYWGWIRQPPGKGLEWIGSISYSGRTYYNPSLKSRVTISVDTSKNQFSLKLSSVTAADTAVYYCARESGYFDYWGQGTLVTVSS

SEQ ID NO: 64 - protein, 108 aa, Artificial Sequence
  Light chain variable region sequence
DIQMTQSPATLSVSPGERATLSCRASQSVGSYLAWYQQKPGQAPRLLIYGASTRATGIPARFSGSGSGTEFTLTISSLQSEDFAVYYCQQYNNWPTWTFGQGTKVEIK

(19) United States
(12) Patent Application Publication
(10) Pub. No.: US 2021/0355195 A1
(43) Pub. Date: Nov. 18, 2021

(54) ANTI-CRIMEAN-CONGO HEMORRHAGIC FEVER VIRUS ANTIBODIES, AND METHODS OF THEIR GENERATION AND USE

(71) Applicants: Albert Einstein College of Medicine, Bronx, NY (US); Adimab, LLC, Lebanon, NH (US); Mapp Biopharmaceutical, Inc., San Diego, CA (US); The University of Texas at Austin, Austin, TX (US)

(72) Inventors: Zachary A. Bornholdt, Encinitas, CA (US); Akaash Mishra, Austin, TX (US); Laura M. Walker, Norwich, VT (US); Noel T. Pauli, Lebanon, NH (US); Daniel Maurer, Cambridge, MA (US); Kartik Chandran, Bronx, NY (US); Jens Maximilian Fels, Bronx, NY (US); Dafna Abelson, San Diego, CA (US); Jason Scott McLellan, Austin, TX (US); Jonathan R. Lai, Bronx, NY (US); Ariel Wirchnianski, Bronx, NY (US); Olivia Vergnolle, Bronx, NY (US)

(21) Appl. No.: 17/308,753

(22) Filed: May 5, 2021

Related U.S. Application Data
(60) Provisional application No. 63/021,004, filed on May 6, 2020.

(51) Int. Cl. C07K 16/10; A61K 45/06; A61K 39/42

(52) U.S. Cl. CPC C07K 16/10; A61K 45/06; C07K 2317/33; C07K 2317/76; A61K 39/42

(57) ABSTRACT

Anti-CCHFV antibodies with neutralizing potency and protective efficacy against CCHFV are provided, as well as methods for their identification, isolation, generation, and methods for their preparation and use are provided.

### CROSS REFERENCE TO RELATED APPLICATIONS

This non-provisional application claims the benefit under 35 U.S.C. 119(e) of U.S. Provisional Application No. 63/021,004, filed May 6, 2020, the whole disclosure of which is incorporated by reference herein.

### STATEMENT REGARDING FEDERALLY FUNDED RESEARCH

This invention was made with government support under grant number U19AI142777 awarded by the National Institutes of Health and under grant number R01AI132246 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 4, 2021, is named 1123-2008US-ST25 and is 41,790 bytes in size.

### FIELD OF THE INVENTION

The invention relates, inter alia, to anti-Crimean-Congo Hemorrhagic Fever Virus (CCHFV) antibodies and functional fragments thereof, and methods and reagents for their preparation and use.

### BACKGROUND OF THE INVENTION

All references cited herein, including without limitation patents, patent applications, and non-patent references and publications referenced throughout are hereby expressly incorporated by reference in their entireties for all purposes.

Crimean-Congo Hemorrhagic Fever Virus (CCHFV) is a widespread tick-borne virus (Nairovirus) of the family Bunyaviridae. Ixodid ticks, such as the Hyalomma tick, are a reservoir and a vector for CCHFV. Wild and domestic animals, such as cattle, goats, sheep and hares, serve as amplifying hosts for the virus. Transmission to humans occurs through contact with infected ticks or animal tissue or blood. CCHF can also be transmitted from one infected human to another by contact with infectious bodily fluids. Documented spread of CCHFV has occurred in hospitals due to improper sterilization of medical equipment, reuse of injection needles, and contamination of medical supplies. According to the Centers for Disease Control and Prevention, CCHFV has a fatality rate of up to 50%. (https://www.cdc.gov/vhf/crimean-congo/symptoms/index.html).

While the pathogenesis of CCHFV is not well understood, a common feature of hemorrhagic fever viruses is their ability to disable the host immune response by rapid replication of the virus along with dysregulation of the vascular system and lymphoid organs. Damage to the endothelium plays an important role in CCHFV pathogenesis and leads to hemostatic failure by stimulating platelet aggregation and degranulation, with subsequent activation of the intrinsic coagulation cascade. Marked pro-inflammatory response disproportional to the extent of lesion is a hallmark feature. Proinflammatory cytokines are key regulators in the pathogenesis and mortality of patients with CCHF. Levels of Interleukin (IL)-6 and Tumor Necrosis Factor (TNF)-α have been shown to be significantly higher in patients with fatal CCHF as compared to those with a non-fatal infection. Ergonul et al., 2006, J Infect Dis. 193(7):941-4.

CCHFV has a tripartite genome comprising a small (S), a medium (M), and a large (L) RNA segment. The M segment encodes two viral glycoproteins, Gn and Gc. Ahmed et al., 2005, J Gen Virology 86:1-10. CCHFV strains may exhibit considerable genetic variability, with the mucin-like domain of Gn being particularly divergent. Gn and Gc are thought to interact with cell surface receptors and mediate the entry of the virus into cells. Bertolotti-Ciarlet et al., 2005, J. Virology, 79(10): 6152-6161. Thus, Gn and Gc serve as potential targets for neutralizing antibodies.

Despite decades of research, the development of safe and effective vaccines or therapeutic and/or prophylactic antibodies against CCHFV has remained elusive, highlighting the need for novel strategies that induce or provide protective immune responses. Indeed, to date there are currently no approved CCHFV vaccines, and treatment of the virus is primarily supportive. Ribavirin is active against CCHFV in vitro, and some groups have shown beneficial effects of ribavirin if given at an early phase of CCHF infection. Tasdelen et al, 2009, Eur J Clin Microbiol Infect Dis. 28(8):929-33. However, a systematic meta-analysis showed no change in mortality rate or difference in length of hospital stay with the use of ribavirin in randomized control studies. Soares-Weiser et al., 2010, BMC Infect Dis. 10:207. Further, the use of ribavirin is limited due to concerns surrounding its potential risk to pregnant women who may be exposed to the aerosolized drug while it is being administered in a hospital environment.

The development of a vaccine is problematic for many reasons, including under reporting and the sporadic nature of the disease. Although neutralizing monoclonal antibodies targeting the CCHFV surface glycoprotein complex (GnGc) have been isolated from immunized mice, the specificities and functional properties of human antibodies elicited by natural CCHFV infection remain unknown. Therefore, there remains a need for highly specific, high affinity, and highly potent neutralizing anti-CCHFV antibodies and antigen-binding fragments thereof.

### SUMMARY OF THE INVENTION

Applicant has discovered, isolated, and characterized, inter alia, an extensive panel of CCHFV-specific monoclonal antibodies from the memory B cells of four CCHFV-convalescent donors. Sequence analysis of these antibodies revealed the GnGc-specific memory B cell repertoires to be highly diverse, with few to no expanded clonal lineages. Binding studies showed that 90% of the antibodies recognize epitopes within the Gc subunit, the putative fusion glycoprotein. Additionally, competitive binding assays revealed that most of these antibodies target one of seven distinct antigenic sites. Approximately half of the antibodies from each donor recognize a single immunodominant site. Neutralization studies performed using a CCHF-VLP assay showed a proportion of antibodies display highly potent neutralizing activity. A subset of these antibodies is currently being evaluated for therapeutic efficacy in a mouse model of CCHF. Altogether, the panel of antibodies described herein provides promising therapeutic candidates and a framework for the rational design of CCHFV vaccines.

Such antibodies may be useful when administered prophylactically (prior to exposure to the virus and infection with the virus) to lessen the severity, or duration of a primary infection with CCHFV, or ameliorate at least one symptom associated with the infection. The antibodies may be used alone or in conjunction with a second agent useful for treating an CCHFV infection. In certain embodiments, the antibodies may be given therapeutically (after exposure to and infection with the virus) either alone, or in conjunction with a second agent to lessen the severity or duration of the primary infection, or to ameliorate at least one symptom associated with the infection. In certain embodiments, the antibodies may be used prophylactically as stand-alone therapy to protect patients who are at risk for acquiring an infection with CCHFV, such as those described above. Any of these patient populations may benefit from treatment with the antibodies of the invention, when given alone or in conjunction with a second agent, including for example, an anti-viral therapy, such as ribavirin, or other anti-viral vaccines.

The antibodies provided herein may be full-length (for example, an IgG1 or IgG4 antibody) or may comprise an antigen-binding portion (for example, a Fab, F(ab′)₂ or scFv fragment), and may be modified to affect functionality, e.g., to eliminate residual effector functions (Reddy et al., (2000), J. Immunol. 164:1925-1933).

Accordingly, in certain embodiments are provided isolated antibodies or antigen-binding fragments thereof that specifically bind to CCHFV, wherein at least one of a CDRH1, a CDRH2, a CDRH3, a CDRL1, a CDRL2, and a CDRL3 amino acid sequence of such antibodies or the antigen-binding fragments thereof are at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to at least one the CDRH1, a CDRH2, a CDRH3, a CDRL1, a CDRL2, and/or a CDRL3 amino acid sequences as disclosed in Table 3 of an antibody selected from Antibody Number 1 through Antibody Number 16 as disclosed in Table 3; and wherein said antibody or the antigen-binding fragment thereof also has one or more of the following characteristics: a) the antibodies or antigen-binding fragments thereof display a clean or low polyreactivity profile; b) the antibodies or antigen-binding fragments thereof display an in vitro neutralization potency (IC₅₀) of between about 0.5 microgram/milliliter (μg/ml) to about 5 μg/ml; between about 0.05 μg/ml to about 0.5 μg/ml; or less than about 0.05 mg/ml; or c) the antibodies or antigen-binding fragments thereof bind at least one of Gc or Gn glycoproteins.

In certain other embodiments, the isolated antibodies or antigen-binding fragments thereof comprise: at least two; or at least three; of the characteristics a) through c) above. Such antibodies may be engineered as bispecific antibodies (abbreviated elsewhere herein as “bsmAbs”) with specificities to complementary epitope sites, for example: a) antibodies targeting epitopes in site I and site III and; b) antibodies targeting epitopes in site I and site VI as defined in FIG. 15A through FIG. 15D. The CCHFV antibodies when combined in such a way enhance the protective efficacy over the individual anitbodies alone or when combined separately as a cocktail of antibodies (See FIGS. 11A-11H, 16A-16I and 17A-17H).

In certain other embodiments, the isolated antibodies or antigen-binding fragments thereof comprise: a) the CDRH1 amino acid sequence of any one of the antibodies designated Antibody Number 1 through Antibody Number 16 as disclosed in Table 3; b) the CDRH2 amino acid sequence of any one of the antibodies designated Antibody Number 1 through Antibody Number 16 as disclosed in Table 3; c) the CDRH3 amino acid sequence of any one of the antibodies designated Antibody Number 1 through Antibody Number 16 as disclosed in Table 3; d) the CDRL1 amino acid sequence of any one of the antibodies designated Antibody Number 1 through Antibody Number 16 as disclosed in Table 3; e) the CDRL2 amino acid sequence of any one of the antibodies designated Antibody Number 1 through Antibody Number 16 as disclosed in Table 3; f) the CDRL3 amino acid sequence of any one of the antibodies designated Antibody Number 1 through Antibody Number 16 as disclosed in Table 3; and/or g) any combination of two or more of a), b), c), d), e), and f).

In certain other embodiments, the isolated antibodies or antigen-binding fragments thereof are selected from the group consisting of antibodies that are at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to any one of the antibodies designated as Antibody Number 1 through Antibody Number 16 as disclosed in Table 3.

In certain other embodiments, the isolated antibodies or antigen-binding fragments thereof comprise: a) a heavy chain (HC) amino acid sequence of any one of the antibodies designated Antibody Number 1 through Antibody Number 16 as disclosed in Table 3; and/or b) a light chain (LC) amino acid sequence of any one of the antibodies designated Antibody Number 1 through Antibody Number 16 as disclosed in Table 3.

The disclosure also contemplates nucleic acids encoding the described anti-CCHF antibodies and expression vectors comprising said nucleic acids as well as host cells or transgenic animals modified to express such antibodies via the nucleic acids and/or expression vectors.

In one embodiment is provided isolated nucleic acid sequences encoding antibodies or antigen-binding fragments thereof disclosed herein.

In other embodiments are provided expression vectors comprising isolated nucleic acid sequences encoding antibodies or antigen-binding fragments disclosed herein.

In other embodiments are provided host cells transfected, transformed, or transduced with nucleic acid sequences encoding antibodies or antigen-binding fragments disclosed herein or expression vectors comprising isolated nucleic acid sequences encoding antibodies or antigen-binding fragments disclosed herein.

In other embodiments are provided pharmaceutical compositions comprising one or more of the isolated antibodies or antigen-binding fragments thereof disclosed herein; and a pharmaceutically acceptable carrier and/or excipient.

In other embodiments are provided pharmaceutical compositions comprising one or more nucleic acid sequences encoding antibodies or antigen-binding fragments disclosed herein, or one or more the expression vectors comprising nucleic acid sequences encoding anitodies or antigen-binding fragments disclosed herein; and a pharmaceutically acceptable carrier and/or excipient.

In other embodiments are provided transgenic organisms comprising nucleic acid sequences encoding antibodies or antigen-binding fragments disclosed herein; or expression vectors comprising nucleic acid sequences encoding antibodies or antigen-binding fragments disclosed herein.

The disclosure further contemplates methods of prevention and/or treatment using the described anti-CCHF antibodies (or nucleic acids encoding or expression vectors comprising such nucleic acids).

In one embodiment is provided methods of treating or preventing a Crimean-Congo Hemorrhagic Fever Virus (CCHFV) infection, or at least one symptom associated with CCHFV infection, comprising administering to a patient in need there of or suspected of being in need thereof: a) one or more antibodies or antigen-binding fragments thereof according to other embodiments disclosed herein; b) one or more nucleic acid sequences encoding antibodies or antigen-binding fragments disclosed herein; an expression vector comprising nucleic acid sequences encoding antibodies or antigen-binding fragments disclosed herein; or a host cell comprising an expression vector comprising nucleic acid sequences encoding antibodies or antigen-binding fragments disclosed herein; or c) a pharmaceutical composition according to other embodiments disclosed herein; such that the CCHFV infection is treated or prevented, or the at least one symptom associated with CCHFV infection is treated, alleviated, or reduced in severity.

In other embodiments the methods further comprise administering to the patient a second therapeutic agent.

In other embodiments the second therapeutic agent is selected group consisting of: an antiviral agent; a vaccine specific for CCHFV; a vaccine specific for influenza virus; an siRNA specific for a CCHFV antigen; and a second antibody specific for a CCHFV antigen.

In certain embodiments are provided pharmaceutical compositions for use in preventing a CCHFV infection in a patient in need thereof or suspected of being in need thereof, or for treating a patient suffering from an CCHFV infection, or for ameliorating at least one symptom or complication associated with the infection, wherein the infection is either prevented, or at least one symptom or complication associated with the infection is prevented, ameliorated, or lessened in severity and/or duration as a result of such use.

In certain embodiments are provided pharmaceutical compositions for use in treating or preventing a CCHFV infection, or at least one symptom associated with said CCHFV infection, in a patient in need thereof or suspected of being in need thereof, wherein the infection is either prevented, or at least one symptom or complication associated with the infection is prevented, ameliorated, or lessened in severity and/or duration as a result of such use.

In certain other embodiments are provided uses of the pharmaceutical compositions in the manufacture of a medicament for preventing a CCHFV infection in a patient in need thereof, or for treating a patient suffering from a CCHFV infection, or for ameliorating at least one symptom or complication associated with the infection, wherein the infection is either prevented, or at least one symptom or complication associated with the infection is prevented, ameliorated, or lessened in severity and/or duration.

In certain other embodiments are provided uses of the pharmaceutical compositions in the manufacture of a medicament for preventing a CCHFV infection, or at least one symptom associated with said CCHFV infection, in a patient in need thereof or suspected of being in need thereof, wherein the infection is either prevented, or at least one symptom or complication associated with the infection is prevented, ameliorated, or lessened in severity and/or duration as a result of such use.

### BRIEF DESCRIPTION OF THE FIGURES

FIG. 1A through FIG. 1C illustrate that GnGc-serum positive CCHF-convalescent donors display GnGc-specific B cell populations. FIG. 1A: Dilution curves show binding of Donor 1 (“CCHF-1”) by GnGc and GP38. FIG. 1B: Dilution curves show binding to a GnGc probe by serum from four CCHF-convalescent donors (Donors 4-7 or “CCHF-4 through CCHF-7”). CCHF-CT-1, CCHF-CT-2, and PBS are controls. FIG. 1C: GnGc-specific B cell sorting. FACS plots show GnGc reactivity of CD19+/CD20+B cells from CCHF-convalescent donors. Numbers beneath plots show total number of IbAr10200 GnGc binding antibodies generated from each donor that had discernable binding.

FIG. 2A and FIG. 2B illustrate that donors show similar proportions of GnGc-specific B cell subsets. FIG. 2A: GnGc-specific B cell sorting. FACS plots show the majority of GnGc-specific B cells were derived from IgM-IgD-, class-switched memory B cells. FIG. 2B: Index sort analysis of surface markers expressed on B cells from four CCHF-convalescent donors.

FIG. 3A through FIG. 3C illustrate how somatic mutation load correlates with time post-infection, CDRH3 lengths are similar to unselected repertoires, and all donors show limited clonal expansion. FIG. 3A: Somatic hypermutation in V_{H} and VK/L. Bar indicates the median number of nucleotide substitutions. Each clonal lineage is only represented once. FIG. 3B: CDRH3 length distribution. CDRH3 length frequencies were compared with unselected adult repertoires. Unselected repertoire data was provided from Larimore et al., (2012) J. Immunol. 189(6) 3221-3230. FIG. 3C: Clonal lineage analysis. Each slice represents one clonal lineage; the size of the slice is proportional to the number of clones in the lineage. The total number of clones is shown in the center of the pie. Clonal lineages were assigned the following criteria: 1) matching H and K/L variable and joining gene segments; 2) identical H and K/L CDR3 loop lengths; and 3) >80% homology in H and K/L CDR3 nucleotide sequences.

FIG. 4A through FIG. 4D illustrate all donors use similar proportions of VH, VK, and VL families. FIG. 4A: Analysis of V_{H} families 1-6. FIG. 4B: Comparison of VK and VL family usage. FIG. 4C: Analysis of VK families 1-5. FIG. 4D: Analysis of VL families 1-7.

FIG. 5 shows memory B cell-derived antibodies have high affinity to GnGc. The fraction of isolated antibodies from each donor that binds to IbAr10200 is shown. Apparent binding affinities are shown for each antibody. Black bars indicate medians.

FIG. 6A through 6D shows the majority of antibodies bind the Gc subunit and cross-react with China and Kosovo derived GnGc. FIG. 6A: Heat map showing Kd values of isolated antibodies. FIG. 6B: Percentage of antibodies from each donor that bind to Gc. FIG. 6C: Percentage of antibodies that bind that bind IbAr only, IbAr/Kosovo, IbAr/China, and IbAr/China/Kosovo. FIG. 6D: Polyreactivity analysis of anti-CCHFV antibodies. The polyreactivity of the isolated anti-CCHFV antibodies was measured using a previously described assay (Xu et al, Protein Eng Des Sel 2013).

FIG. 7 shows the binding affinities of antibodies that bind GnGc but not Gc. Plots show Kd values for: 1) antibodies from the ADI-36120 bin which bind to IbAr10200, China, and Kosovo strains, and 2) other antibodies from undefined bins which bind to some, but not all, of the CCHFV strains. WB/NB—weak or non-binders. PF—Poor fit, where a response is high but curve fit is poor making it hard to determine an accurate Kd value.

FIG. 8A and FIG. 8B illustrate most antibodies from each donor bin with ADI-36193. FIG. 8A: FACS plots on the top row show antibody binding with only antigen present (top left) and disrupted binding with the addition of a competitor antibody (top right). FACS plots on the bottom row show no effects of a non-competitor antibody in either condition. FIG. 8B: Percentage of antibodies that fall within different bins across donors. An antibody falls within a bin if the fold reduction=(antigen (Ag) Binding_{Ag}/Antibody_{Ag})/(Ag Binding_{Fab+Ag}/Antibody_{Fab+Ag}) >10.

FIG. 9A through FIG. 9F show antibodies in different bins have germline preferences. FIG. 9A: Heat map of V_{H} and V_{L} germline gene usage for all antibodies of bin ADI-36193 for which at least one VH/V_{L} pairing was used in ≥0.5% of the antibodies of bin ADI-36193. FIG. 9B: Heat map of V_{H} and V_{L} germline gene usage for all antibodies of bin ADI-36121 for which at least one VH/V_{L} pairing was used in ≥0.5% of the antibodies of bin ADI-36121. FIG. 9C: Heat map of V_{H} and V_{L} germline gene usage for all antibodies of bin ADI-36122 for which at least one VH/V_{L} pairing was used in ≥0.5% of the antibodies of bin ADI-36122. FIG. 9D: Heat map of V_{H} and V_{L} germline gene usage for all antibodies of bin ADI-36121/36125 for which at least one VH/V_{L} pairing was used in ≥0.5% of the antibodies of bin ADI-36121/36125. FIG. 9E: Heat map of V_{H} and V_{L} germline gene usage for all antibodies of bin ADI-36125 for which at least one VH/V_{L} pairing was used in ≥0.5% of the antibodies of bin ADI-36125. FIG. 9F: Heat map of V_{H} and V_{L} germline gene usage for all antibodies of bin ADI-36120 for which at least one VH/V_{L} pairing was used in ≥0.5% of the antibodies of bin ADI-36120.

FIG. 10A through FIG. 10D show ADI-36193 and ADI-36121 bin antibodies have the highest proportion of neutralizing antibodies. FIG. 10A: Antibodies from each donor that show neutralization potency. Black bars indicate medians. FIG. 10B: Neutralization potency shown for antibodies sorted by cell marker. Black bars indicate medians. FIG. 10C: The percentage of antibodies with >70, >50, >30, >10, and <10 percent neutralization for each donor is shown. FIG. 10D: The percentage of antibodies with >70, >50, >30, >10, and <10 percent neutralization for each antibody bin is shown.

FIG. 11A through FIG. 11H illustrate neutralization curves and combination index analysis for ADI-36121 and four different 36193 bin members, as well as each parental antibody alone. FIG. 11A: Neutralization curves of ADI-36121, ADI-37801, and 1:1 molar ratio combinations of ADI-36121 plus ADI-37801. FIG. 11B: Neutralization curves of ADI-36121, ADI-37817, and 1:1 molar ratio combinations of ADI-36121 plus ADI-37817. FIG. 11C: Neutralization curves of ADI-36121, ADI-37836, and 1:1 molar ratio combinations of ADI-36121 plus ADI-37836. FIG. 11D: Neutralization curves of ADI-36121, ADI-37842, and 1:1 molar ratio combinations of ADI-36121 plus ADI-37842. FIG. 11E: Combination index analysis for ADI-36121 plus ADI-37801. FIG. 11F: Combination index analysis for ADI-36121 plus ADI-37817. FIG. 11G: Combination index analysis for ADI-36121 plus ADI-37836. FIG. 11H: Combination index analysis for ADI-36121 plus ADI-37842.

ADI-37801 displays potent synergistic neutralization with ADI-36121 and ADI-36145. (A) Neutralization curves of ADI-36121, ADI-36145, and a 1:1 combination of the two mAbs. (B) Neutralization curves of ADI-36121, ADI-37801, and a 1:1 combination of the two mAbs. (C) Neutralization curves of ADI-36145, ADI-37801, and a 1:1 combination of the two mAbs. (D) Combination index analysis of ADI-36121 and ADI-36145 neutralization, showing a CI of ˜1 across all effect sizes indicating additive neutralization. (E) Combination index analysis of ADI-36121 and ADI-37801 neutralization, showing a CI <1 at effect sizes over 40% neutralization which indicates synergistic neutralization. (F) Combination index analysis of ADI-36145 and ADI-37801 neutralization, showing a CI <1 at effect sizes over 40% neutralization which indicates synergistic neutralization. (G) Summary of CI values at 50% neutralization for mAb combinations against tecVLPs carrying GnGc from four strains of CCHFV.

FIG. 12 illustrates a methodology for epitope mapping for anti-Gc antibodies. The methodology may include creating an alanine (ala) scanning library, expressing the Gc protein on a yeast surface, sorting for loss of binding, and sequencing to determine epitope.

FIG. 13A and FIG. 13B illustrate Gc expressed on the surface of yeast is conformationally intact. FIG. 13A: FACS sorting shows all anti-Gc binning antibodies bind yeast surface-displayed Gc. FIG. 13B: Plot showing mean fluorescence intensity (MFI) for anti-Gc binning antibodies over concentration.

FIG. 14A shows survival curves for mice challenged with Turkey2004 and treated with a single 250 μg dose of the indicated mAb 30 minutes post-infection. **, Mantel-Cox P<0.01. FIG. 14B are clinical scores of animals within the study cohort are shown.

FIG. 15A shows a heat map depicting the magnitude of loss of binding compared to wild type when stained with single mutant clones. Darker shade indicates a greater loss of binding. For some residues with multiple mutations, averages of binding loss for a mutation at a given position are shown. * denotes the introduction of potential N-linked glycosylation sites. FIG. 15B is an antigenic site mapped on the surface of one CCHFV Gc protomer within the postfusion trimer. The trimer axis is shown in light blue. The trimer interface is outlined in black (right). All residues from FIG. 15A that are not highlighted in FIG. 15B are occluded from the surface. FIG. 15C shows sequence similarity across 15 representative CCHFV strains and FIG. 15D shows sequence similarity across 14 orthonairoviruses. IC indicates the nairovirus-specific insertions cluster.

FIG. 16A shows that eEngineered dual variable domain IgGs provide enhanced synergistic neutralization. Neutralization curves against (A) Oman tecVLPs (FIG. 16B), (B) IbAr10200 tecVLPs (FIG. 16C), (C) Turkey tecVLPs (FIG. 16D), (D) Kosova Hoti tecVLPs, for ADI-36121, ADI-37801 (FIG. 16E), and corresponding combinations and DVDs. Neutralization curves against (E) Oman tecVLPs (FIG. 16F), (F) IbAr10200 tecVLPs (FIG. 16G), (G) Turkey tecVLPs (FIG. 16H), (H) Kosova Hoti tecVLPs (FIG. 16I), for ADI-36145, ADI-37801 (FIG. 16I), and corresponding combinations and DVDs.

FIG. 17A through FIG. 17H shows that DVD-121-801 provides therapeutic protection against CCHFV challenge. (A) Survival curves of mice treated with ADI-36121 or ADI-36145 30 min post challenge with CCHFV Turkey2004 (FIG. 17A) and (B) associated changes in weight (FIG. 17B). (C) Survival curves of mice treated with ADI-36121, ADI-36145, ADI-37801, equimolar combinations thereof, or c13G8 1 day prior to challenge with CCHFV IbAr10200 (FIG. 17C), and (D) associated changes in weight (FIG. 17D). (E) Survival curves of mice treated with ADI-36121, ADI-36145, ADI-37801, equimolar combinations thereof, or c13G8 1 day after challenge with CCHFV IbAr10200 (FIG. 17E), and (F) associated changes in weight (FIG. 17F). (G) Survival curves of mice treated with ADI-36121, equimolar combinations of ADI-36121/36145 and ADI-37801, DVD-121-801, or DVD-145-801 1 day after challenge with CCHFV IbAr10200 (FIG. 17G), and (H) associated changes in weight (FIG. 17H).

### DETAILED DESCRIPTION OF THE INVENTION

Unless defined otherwise, all technical and scientific terms used herein have the same meaning as commonly understood by one of ordinary skill in the art to which this invention belongs.

### Definitions

“Crimean-Congo Hemorrhagic Fever Virus”, also referred to as “CCHFV” or “CCHF”, is an RNA virus typically spread by tick bites, contact with animal tissue carrying the virus, or contact with body fluids of persons carrying the virus.

The term “GnGc” refers to the CCHFV surface glycoprotein complex comprising membrane glycoproteins Gn and Gc.

The term “IbAr10200” or “Ibar” refers to the CCHFV strain originally isolated from Hyalomma excavatum ticks from Sokoto, Nigeria.

The term “IC₅₀” refers to the “half maximal inhibitory concentration”, which value measures the effectiveness of compound (e.g., anti-CCHFV antibody) inhibition towards a biological or biochemical utility. This quantitative measure indicates the quantity required for a particular inhibitor to inhibit a given biological process by half. In certain embodiments, CCHFV neutralization potencies for anti-CCHFV neutralizing antibodies disclosed herein are expressed as neutralization ICso values.

As used herein, the terms “treat,” “treatment” and “treating” refer to the reduction or amelioration of the progression, severity, and/or duration of a CCHFV infection, or a symptom or condition related thereto (such as fever, muscle pains, headaches, vomiting, diarrhea, bleeding, or a combination thereof) resulting from the administration of one or more therapies (including, but not limited to, the administration of one or more prophylactic or therapeutic agents). In certain embodiments, such terms refer to the reduction or inhibition of the replication of CCHFV, the inhibition or reduction in the spread of CCHFV to other tissues or subjects, the inhibition or reduction of infection of a cell with CCHFV, or the amelioration of one or more symptoms associated with a CCHFV infection.

As used herein, the terms “prevent,” “preventing,” and “prevention” refer to the prevention or inhibition of the development or onset of a CCHFV infection or condition related thereto in a subject, the prevention or inhibition of the progression of a CCHFV infection or a condition related thereto resulting from the administration of a therapy (e.g., a prophylactic or therapeutic agent), the prevention of a symptom of a CCHFV infection or condition related thereto, or the administration of a combination of therapies (e.g., a combination of prophylactic or therapeutic agents). As used herein, the terms “ameliorate” and “alleviate” refer to a reduction or diminishment in the severity a condition or any symptoms thereof.

The term “antibody” (or “Ab”), as used herein, is intended to refer to immunoglobulin molecules comprised of four polypeptide chains, two heavy (H) chains and two light (L) chains interconnected by disulfide bonds (i.e., “full antibody molecules”), as well as multimers thereof (e.g., IgM) or antigen-binding fragments thereof. Each heavy chain is comprised of a heavy chain variable region (“HCVR” or “V_{H}”) and a heavy chain constant region (comprised of domains C_{H}1, C_{H}2, and C_{H}3). Each light chain is comprised of a light chain variable region (“LCVR or “V_{L}”) and a light chain constant region (C_{L}). The V_{H} and V_{L} regions can be further subdivided into regions of hypervariability, termed complementarity determining region (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) may be identical to the human germline sequences, or may be naturally or artificially modified. An amino acid consensus sequence may be defined based on a side-by-side analysis of two or more CDRs. Accordingly, the CDRs in a heavy chain are designated “CHRH1”, “CDRH2”, and “CDRH3”, respectively, and the CDRs in a light chain are designated “CDRL1”, “CDRL2”, and “CDRL3”.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically.

The fully human monoclonal antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes fully monoclonal antibodies comprising variants of any of the CDR amino acid sequences disclosed herein having one or more conservative substitutions. For example, the present invention includes antibodies having CDR amino acid sequences with, e.g., 10 or fewer, 8 or fewer, 6 or fewer, 4 or fewer, etc. conservative amino acid substitutions relative to any of the CDR amino acid sequences disclosed herein.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3.

However, the term “human antibody”, as used herein, is not intended to include mAbs in which CDR sequences derived from the germline of another mammalian species (e.g., mouse), have been grafted onto human FR sequences.

The term “humanized antibody” refers to human antibody in which one or more CDRs of such antibody have been replaced with one or more corresponding CDRs obtained a non-human derived (e.g., mouse, rat, rabbit, primate) antibody. Humanized antibodies may also include certain non-CDR sequences or residues derived from such non-human antibodies as well as the one or more non-human CDR sequence. Such antibodies may also be referred to as “chimeric” antibodies.

The term “recombinant” generally refers to any protein, polypeptide, or cell expressing a gene of interest that is produced by genetic engineering methods. The term “recombinant” as used with respect to a protein or polypeptide, means a polypeptide produced by expression of a recombinant polynucleotide. The proteins used in the immunogenic compositions of the invention may be isolated from a natural source or produced by genetic engineering methods.

The antibodies of the invention may, in some embodiments, be recombinant human antibodies. The term “recombinant human antibody”, as used herein, is intended to include all antibodies, including human or humanized antibodies, that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, biolayer interferometry measurements using, e.g., a ForteBio Octet HTX instrument (Pall Life Sciences), which bind specifically to CCHFV. Moreover, multi-specific antibodies that bind to CCHFV protein and one or more additional antigens, or a bi-specific that binds to two different regions of CCHFV are nonetheless considered antibodies that “specifically bind”, as used herein. In certain embodiments, the antibodies disclosed herein display equilibrium dissociation constants (and hence specificities) of about 1×10⁻⁶ M; about 1×10⁻⁷ M; about 1×10⁻⁸ M; about 1×10⁻⁹ M; about 1×10⁻¹⁰ M; between about 1×10⁻⁶ M and about 1×10⁻⁷ M; between about 1×10⁻⁷ M and about 1×10⁻⁸ M; between about 1×10⁻⁸ M and about 1×10⁻⁹ M; between about 1×10⁻⁹ M and about 1×10⁻¹⁰ M; or between about 1×10⁻⁹ M and about 1×10⁻¹⁰ M.

The term “high affinity” antibody refers to those mAbs having a binding affinity to CCHFV, expressed as KD, of at least 10⁻⁹ M; more preferably 10−¹⁰ M, more preferably 10⁻¹¹ M, more preferably 10⁻¹²M as measured by surface plasmon resonance, e.g., BIACORE™, biolayer interferometry measurements using, e.g., a ForteBio Octet HTX instrument (Pall Life Sciences), or solution-affinity ELISA.

By the term “slow off rate”, “Koff” or “kd” is meant an antibody that dissociates from CCHFV, with a rate constant of 1×10⁻³ s⁻¹ or less, preferably 1×10⁻⁴ s⁻¹ or less, as determined by surface plasmon resonance, e.g., BIACORE™ or a ForteBio Octet HTX instrument (Pall Life Sciences).

The terms “antigen-binding portion”, “antigen-binding fragment”, and the like, as used herein, include any naturally occurring, enzymatically obtainable, synthetic, or genetically engineered polypeptide or glycoprotein that specifically binds an antigen to form a complex. In certain embodiments, the terms “antigen-binding portion” or “antibody fragment”, as used herein, refer to one or more fragments of an antibody that retains the ability to bind to CCHFV.

An antibody fragment may include a Fab fragment, a F(ab′)₂ fragment, a Fv fragment, a dAb fragment, a fragment containing a CDR, or an isolated CDR. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1 ; (ii) V_{H}-C_{H}2; (iii) V_{H}-C_{H}3; (iv) V_{H}-C_{H}1 -CH2; (V) V_{H}-C_{H}1-C_{H}2-C_{H}3; (vi) V_{H}-C_{H}2-C_{H}3; (vii) V_{H}-C_{L}; (viii) V_{L}-C_{H}1 ; (1X) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

The specific embodiments, antibody or antibody fragments of the invention may be conjugated to a therapeutic moiety (“immunoconjugate”), such as an antibiotic, a second anti-CCHFV antibody, a vaccine, or a toxoid, or any other therapeutic moiety useful for treating a CCHFV infection.

An “isolated antibody”, as used herein, refers to an antibody that is substantially free of other antibodies having different antigenic specificities (e.g., an isolated antibody that specifically binds CCHFV, or a fragment thereof, is substantially free of Abs that specifically bind antigens other than CCHFV).

A “blocking antibody” or a “neutralizing antibody”, as used herein (or an “antibody that neutralizes CCHFV activity”), refers to an antibody whose binding to an antigen, e.g., a CCHFV antigen as the case may be as disclosed herein, results in inhibition of at least one biological activity of the target, e.g., CCHFV. For example, an antibody of the invention may aid in blocking the fusion of CCHFV to a host cell, or prevent syncytia formation, or prevent the primary disease caused by CCHFV. Alternatively, an antibody of the invention may demonstrate the ability to ameliorate at least one symptom of the CCHFV infection. This inhibition of the biological activity of CCHFV can be assessed by measuring one or more indicators of CCHFV biological activity by one or more of several standard in vitro assays (such as a neutralization assay, as described herein) or in vivo assays known in the art (for example, animal models to look at protection from challenge with CCHFV following administration of one or more of the antibodies described herein).

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity”, or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. Accordingly, nucleic acid sequences that display a certain percentage “identity” share that percentage identity, and/or are that percentage “identical” to one another. A nucleic acid molecule having substantial identity to a reference nucleic acid molecule may, in certain instances, encode a polypeptide having the same or substantially similar amino acid sequence as the polypeptide encoded by the reference nucleic acid molecule.

In certain embodiments, the disclosed antibody nucleic acid sequences are, e.g.: at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to other sequences and/or share such percentage identities with one another (or with certain subsets of the herein-disclosed antibody sequences).

As applied to polypeptides, the term “substantial identity” or “substantially identical” means that two peptide sequences, when optimally aligned, such as by the programs GAP or BESTFIT using default gap weights, share at least 90% sequence identity, even more preferably at least 95%, 98% or 99% sequence identity. Accordingly, amino acid sequences that display a certain percentage “identity” share that percentage identity, and/or are that percentage “identical” to one another. Accordingly, amino acid sequences that display a certain percentage “identity” share that percentage identity, and/or are that percentage “identical” to one another.

In certain embodiments, the disclosed antibody amino acid sequences are, e.g.,: at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to other sequences and/or share such percentage identities with one another (or with certain subsets of the herein-disclosed antibody sequences).

Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. (See, e.g., Pearson (1994) Methods Mol. Biol. 24: 307-331). Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-45. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. (See, e.g., Altschul et al. (1990) J. Mol. Biol. 215: 403 410 and (1997) Nucleic Acids Res. 25:3389 402).

In certain embodiments, the antibody or antibody fragment for use in the method of the invention may be mono-specific, bi-specific, or multi-specific. Multi-specific antibodies may be specific for different epitopes of one target polypeptide or may contain antigen-binding domains specific for epitopes of more than one target polypeptide.

By the phrase “therapeutically effective amount” is meant an amount that produces the desired effect for which it is administered. The exact amount will depend on the purpose of the treatment and will be ascertainable by one skilled in the art using known techniques (see, for example, Lloyd (1999) The Art, Science and Technology of Pharmaceutical Compounding).

### Preparation of Human Antibodies

As disclosed herein, anti-CCHFV antibodies may be obtained from human B cells using techniques available to the artisan, and, for example, as described in the EXAMPLES below. Methods for generating human antibodies in transgenic animals, such as mice, are also known in the art and may be employed in order to derive antibodies in accordance with the present disclosure. Any such known methods can be used in the context of the present invention to make human antibodies that specifically bind to CCHFV (see, for example, U.S. Pat. No. 6,596,541).

In certain embodiments, the antibodies of the instant invention possess affinities (K_{D}) ranging from about 1.0×10−⁷M to about 1.0×10⁻¹²M, when measured by binding to antigen either immobilized on solid phase or in solution phase. In certain embodiments, the antibodies of the invention possess affinities (K_{D}) ranging from about 1×10⁻⁷ M to about 6 x10⁻¹⁰M, when measured by binding to antigen either immobilized on solid phase or in solution phase. In certain embodiments, the antibodies of the invention possess affinities (K_{D}) ranging from about 1×10⁻⁷ M to about 9×10⁻¹⁰M, when measured by binding to antigen either immobilized on solid phase or in solution phase.

In addition to the specific anti-CCHFV antibodies and antibody fragments disclosed herein, the present disclosure also contemplates variants of those antibodies and antibody fragemnts that maintain bioequivalency. Such variant antibodies and antibody fragments comprise one or more additions, deletions, or substitutions of amino acids when compared to parent sequence but exhibit biological activity that is essentially equivalent to that of the described antibodies. Likewise, the antibody-encoding DNA sequences of the present invention encompass sequences that comprise one or more additions, deletions, or substitutions of nucleotides when compared to the disclosed sequence, but that encode an antibody or antibody fragment that is essentially bioequivalent to an antibody or antibody fragment of the invention.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Biological and Biophysical Characteristics of the Antibodies

In certain embodiments, the inventive antibodies and antigen-binding fragments thereof specifically bind to CCHFV, wherein at least one of the CDRH1, CDRH2, CDRH3, CDRL1, CDRL2, and/or CDRL3 amino acid sequences is at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to the corresponding CDRH1, CDRH2, CDRH3, CDRL1, CDRL2, and/or CDRL3 amino acid sequence as disclosed in Table 3 of an antibody selected from Antibody Number 1 through Antibody Number 16 as disclosed in Table 3.

In certain other embodiments, the inventive antibodies and antigen-binding fragments thereof advantageously display a clean or low polyreactivity profile (see, e.g., WO 2014/179363 and Xu et al., Protein Eng Des Sel, Oct; 26(10):663-70. doi: 10.1093/protein/gzt047), and are thus particularly amenable to development as safe, efficacious, and developable therapeutic and/or prophylactic anti-CCHFV treatments.

In certain embodiments, the inventive antibodies and antigen-binding fragments thereof display an in vitro neutralization potency (IC₅₀) of between about 0.5 microgram/milliliter (μg/ml) to about 5 μg/ml; between about 0.05 μg/ml to about 0.5 μg/ml; or less than about 0.05 mg/ml.

In certain embodiments, the inventive antibodies and antigen-binding fragments thereof comprise the CDRH3 amino acid sequence of any one of the antibodies designated Antibody Number 1 through Antibody Number 16 as disclosed in Table 3.

In certain embodiments, the inventive antibodies and antigen-binding fragments thereof comprise the CDRH2 amino acid sequence of any one of the antibodies designated Antibody Number 1 through Antibody Number 16 as disclosed in Table 3.

In certain embodiments, the inventive antibodies and antigen-binding fragments thereof comprise the CDRH1 amino acid sequence of any one of the antibodies designated Antibody Number 1 through Antibody Number 16 as disclosed in Table 3.

In certain embodiments, the inventive antibodies and antigen-binding fragments thereof comprise the CDRL3 amino acid sequence of any one of the antibodies designated Antibody Number 1 through Antibody Number 16 as disclosed in Table 3.

In certain embodiments, the inventive antibodies and antigen-binding fragments thereof comprise the CDRL2 amino acid sequence of any one of the antibodies designated Antibody Number 1 through Antibody Number 16 as disclosed in Table 3.

In certain embodiments, the inventive antibodies and antigen-binding fragments thereof comprise the CDRL1 amino acid sequence of any one of the antibodies designated Antibody Number 1 through Antibody Number 16 as disclosed in Table 3.

In certain embodiments, the inventive antibodies and antigen-binding fragments thereof comprise any combination of two, three, four, five, or six characteristics disclosed in the immediately preceding six paragraphs.

In certain embodiments, the inventive antibodies and antigen-binding fragments thereof comprise a heavy chain (HC) amino acid sequence of any one of the antibodies designated Antibody Number 1 through Antibody Number 16 as disclosed in Table 3. In certain embodiments, the inventive antibodies and antigen-binding fragments thereof comprise a light chain (LC) amino acid sequence of any one of the antibodies designated Antibody Number 1 through Antibody Number 16 as disclosed in Table 3. In certain embodiments, the inventive antibodies and antigen-binding fragments thereof comprise a heavy chain (HC) amino acid sequence and a light chain (LC) amino acid sequence of any one of the antibodies designated Antibody Number 1 through Antibody Number 16 as disclosed in Table 3.

In certain embodiments, the inventive antibodies and antigen-binding fragments thereof are each selected from the group consisting antibodies that are at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to any one of the antibodies designated as Antibody Number 1 through Antibody Number 16 as disclosed in Table 3.

In certain embodiments, the inventive antibodies and antigen-binding fragments thereof comprise are each selected from the group consisting of the antibodies designated as Antibody Number 1 through Antibody Number 16 as disclosed in Table 3.

In certain embodiments, isolated nucleic acid sequences are provided that encode antibodies that specifically bind to CCHFV and antigen-binding fragments thereof, wherein at least one of the CDRH1, CDRH2, CDRH3, CDRL1, CDRL2, and/or CDRL3 amino acid sequences of the antibody or the antigen-binding fragment thereof is at least 70% identical; at least 75% identical; 80% identical; at least 85% identical; at least 90% identical; at least 95% identical; at least 96% identical; at least 97% identical; at least 98% identical; at least 99%; and/or all percentages of identity in between; to at least one the CDRH1, CDRH2, CDRH3, CDRL1, CDRL2, and/or CDRL3 amino acid sequences as disclosed in Table 3 of an antibody selected from Antibody Number 1 through Antibody Number 16 as disclosed in Table 3. In certain embodiments, such nucleic acid sequences are selected from those nucleic acid sequences that are disclosed in Table 3, and compliments thereof.

In certain embodiments, isolated nucleic acid sequences are provided that encode the inventive antibodies and antigen-binding fragments thereof, wherein such nucleic acid sequences comprise sequences that encode the CDRH3 amino acid sequence of the antibodies designated Antibody Number 1 through Antibody Number 16 as disclosed in Table 3. In certain embodiments, such nucleic acid sequences are selected from those nucleic acid sequences that are disclosed in Table 3, and compliments thereof.

In certain embodiments, isolated nucleic acid sequences are provided that encode the inventive antibodies and antigen-binding fragments thereof, wherein such nucleic acid sequences comprise sequences that encode the CDRH2 amino acid sequences of the antibodies designated Antibody Number 1 through Antibody Number 16 as disclosed in Table 3. In certain embodiments, such nucleic acid sequences are selected from those nucleic acid sequences that are disclosed in Table 3, and compliments thereof.

In certain embodiments, isolated nucleic acid sequences are provided that encode the inventive antibodies and antigen-binding fragments thereof, wherein such nucleic acid sequences comprise sequences that encode the CDRH1 amino acid sequences of the antibodies designated Antibody Number 1 through Antibody Number 16 as disclosed in Table 3. In certain embodiments, such nucleic acid sequences are selected from those nucleic acid sequences that are disclosed in Table 3, and compliments thereof.

In certain embodiments, isolated nucleic acid sequences are provided that encode the inventive antibodies and antigen-binding fragments thereof, wherein such nucleic acid sequences comprise sequences that encode the CDRL3 amino acid sequences of the antibodies designated Antibody Number 1 through Antibody Number 16 as disclosed in Table 3. In certain embodiments, such nucleic acid sequences are selected from those nucleic acid sequences that are disclosed in Table 3, and compliments thereof.

In certain embodiments, isolated nucleic acid sequences are provided that encode the inventive antibodies and antigen-binding fragments thereof, wherein such nucleic acid sequences comprise sequences that encode the CDRL2 amino acid sequences of the antibodies designated Antibody Number 1 through Antibody Number 16 as disclosed in Table 3. In certain embodiments, such nucleic acid sequences are selected from those nucleic acid sequences that are disclosed in Table 3, and compliments thereof.

In certain embodiments, isolated nucleic acid sequences are provided that encode the inventive antibodies and antigen-binding fragments thereof, wherein such nucleic acid sequences comprise sequences that encode the CDRL1 amino acid sequences of the antibodies designated Antibody Number 1 through Antibody Number 16 as disclosed in Table 3. In certain embodiments, such nucleic acid sequences are selected from those nucleic acid sequences that are disclosed in Table 3, and compliments thereof.

In certain embodiments, isolated nucleic acid sequences are provided that encode the inventive antibodies and antigen-binding fragments thereof, wherein such nucleic acid sequences comprise sequences that encode the heavy chain (HC) amino acid sequences of the antibodies designated Antibody Number 1 through Antibody Number 16 as disclosed in Table 3. In certain embodiments, such nucleic acid sequences are selected from those nucleic acid sequences that are disclosed in Table 3, and compliments thereof.

In certain embodiments, isolated nucleic acid sequences are provided that encode the inventive antibodies and antigen-binding fragments thereof, wherein such nucleic acid sequences comprise sequences that encode the heavy chain (LC) amino acid sequences of the antibodies designated Antibody Number 1 through Antibody Number 16 as disclosed in Table 3. In certain embodiments, such nucleic acid sequences are selected from those nucleic acid sequences that are disclosed in Table 3, and compliments thereof.

### Epitope Binning and Related Technologies

As described above and as demonstrated in the EXAMPLES, Applicant has characterized the epitopic binning of the inventive antibodies and antigen-binding fragments thereof. In addition to the methods for conducting such characterization, various other techniques are available to the artisan that can be used to carry out such characterization or to otherwise ascertain whether an antibody “interacts with one or more amino acids” within a polypeptide or protein. Exemplary techniques include, for example, a routine cross-blocking assay such as that described Antibodies, Harlow and Lane (Cold Spring Harbor Press, Cold Spring Harb., NY) can be performed. Other methods include alanine scanning mutational analysis, peptide blot analysis (Reineke (2004) Methods Mol Biol 248:443-63), peptide cleavage analysis crystallographic studies and NMR analysis. In addition, methods such as epitope excision, epitope extraction and chemical modification of antigens can be employed (Tomer (2000) Protein Science 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues that correspond to the specific amino acids with which the antibody interacts. See, e.g., Ehring (1999) Analytical Biochemistry 267 (2):252-259; Engen and Smith (2001) Anal. Chem. 73 :256A-265A.

As the artisan will understand, an epitope can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (US 2004/0101920). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the invention into groups of antibodies binding different epitopes.

As the artisan understands, one can easily determine whether an antibody binds to the same epitope as, or competes for binding with, a reference anti-CCHFV antibody by using routine methods available in the art. For example, to determine if a test antibody binds to the same epitope as a reference CCHFV antibody of the invention, the reference antibody is allowed to bind to a CCHFV protein or peptide under saturating conditions. Next, the ability of a test antibody to bind to the CCHFV molecule is assessed. If the test antibody is able to bind to CCHFV following saturation binding with the reference anti-CCHFV antibody, it can be concluded that the test antibody binds to a different epitope than the reference anti-CCHFV antibody. On the other hand, if the test antibody is not able to bind to the CCHFV molecule following saturation binding with the reference anti-CCHFV antibody, then the test antibody may bind to the same epitope as the epitope bound by the reference anti-CCHFV antibody of the invention.

To determine if an antibody competes for binding with a reference anti-CCHFV antibody, the above-described binding methodology is performed in two orientations: In a first orientation, the reference antibody is allowed to bind to a CCHFV molecule under saturating conditions followed by assessment of binding of the test antibody to the CCHFV molecule. In a second orientation, the test antibody is allowed to bind to a CCHFV molecule under saturating conditions followed by assessment of binding of the reference antibody to the CCHFV molecule. If, in both orientations, only the first (saturating) antibody is capable of binding to the CCHFV molecule, then it is concluded that the test antibody and the reference antibody compete for binding to CCHFV. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. (1990) 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Immunoconjugates

The invention encompasses a human CCHFV monoclonal antibody conjugated to a therapeutic moiety (“immunoconjugate”), such as an agent that is capable of reducing the severity of primary infection with CCHFV, or to ameliorate at least one symptom associated with CCHFV infection, including fever, muscle pains, headache, vomiting, diarrhea, bleeding, or the severity thereof. Such an agent may be a second different antibody to CCHFV, or a vaccine. The type of therapeutic moiety that may be conjugated to the anti-CCHFV antibody and will take into account the condition to be treated and the desired therapeutic effect to be achieved. Alternatively, if the desired therapeutic effect is to treat the sequelae or symptoms associated with CCHFV infection, or any other condition resulting from such infection, such as, but not limited to, disseminated intravascular coagulation, acute kidney failure, and acute respiratory distress syndrome, it may be advantageous to conjugate an agent appropriate to treat the sequelae or symptoms of the condition, or to alleviate any side effects of the antibodies of the invention. Examples of suitable agents for forming immunoconjugates are known in the art, see for example, WO 05/103081.

### Multi-Specific Antibodies

The antibodies of the present invention may be mono-specific, bi-specific, or multi-specific. Multi-specific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The antibodies of the present invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multi-specific antibody with a second binding specificity.

### Therapeutic Administration and Formulations

The invention provides therapeutic compositions comprising the inventive anti-CCHFV antibodies or antigen-binding fragments thereof. The administration of therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of each of the antibodies of the invention may vary depending upon the age and the size of a subject to be administered, target disease, conditions, route of administration, and the like. When the antibodies of the present invention are used for treating a CCHFV infection, or for treating one or more symptoms associated with a CCHFV infection, such as the fever, diarrhea, or bleeding associated with a CCHFV infection in a patient, or for lessening the severity of the disease, it is advantageous to administer each of the antibodies of the present invention intravenously or subcutaneously normally at a single dose of about 0.01 to about 30 mg/kg body weight, more preferably about 0.1 to about 20 mg/kg body weight, or about 0.1 to about 15 mg/kg body weight, or about 0.02 to about 7 mg/kg body weight, about 0.03 to about 5 mg/kg body weight, or about 0.05 to about 3 mg/kg body weight, or about 1 mg/kg body weight, or about 3.0 mg/kg body weight, or about 10 mg/kg body weight, or about 20 mg/kg body weight. Multiple doses may be administered as necessary. Depending on the severity of the condition, the frequency and the duration of the treatment can be adjusted. In certain embodiments, the antibodies or antigen-binding fragments thereof of the invention can be administered as an initial dose of at least about 0.1 mg to about 800 mg, about 1 to about 600 mg, about 5 to about 300 mg, or about 10 to about 150 mg, to about 100 mg, or to about 50 mg. In certain embodiments, the initial dose may be followed by administration of a second or a plurality of subsequent doses of the antibodies or antigen-binding fragments thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings {e.g., oral mucosa, nasal mucosa, rectal and intestinal mucosa, etc.) and may be administered together with other biologically active agents. Administration can be systemic or local. It may be delivered as an aerosolized formulation (See US201 1/031 1515 and US2012/0128669). The delivery of agents useful for treating respiratory diseases by inhalation is becoming more widely accepted (See A. J. Bitonti and J. A. Dumont, (2006), Adv. Drug Deliv. Rev, 58:1 106-1 1 18). In addition to being effective at treating local pulmonary disease, such a delivery mechanism may also be useful for systemic delivery of antibodies (See Maillet et al. (2008), Pharmaceutical Research, Vol. 25, No. 6, 2008).

The pharmaceutical composition can be also delivered in a vesicle, in particular a liposome (see, for example, Langer (1990) Science 249:1527-1533).

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUIIVIALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but certainly are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.) and the HUMIRA™ Pen (Abbott Labs, Abbott Park, Ill.), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Administration Regimens

According to certain embodiments, multiple doses of an antibody to CCHFV may be administered to a subject over a defined time course. The methods according to this aspect of the invention comprise sequentially administering to a subject multiple doses of an antibody to CCHFV. As used herein, “sequentially administering” means that each dose of antibody to CCHFV is administered to the subject at a different point in time, e.g., on different days separated by a predetermined interval (e.g., hours, days, weeks or months). The present invention includes methods which comprise sequentially administering to the patient a single initial dose of an antibody to CCHFV, followed by one or more secondary doses of the antibody to CCHFV and optionally followed by one or more tertiary doses of the antibody to CCHFV.

The terms “initial dose,” “secondary doses,” and “tertiary doses,” refer to the temporal sequence of administration of the antibody to CCHFV. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of antibody to CCHFV, but generally may differ from one another in terms of frequency of administration. In certain embodiments, however, the amount of antibody to CCHFV contained in the initial, secondary and/or tertiary doses vary from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present invention, each secondary and/or tertiary dose is administered 1 to 26 (e.g., 1, 1½, 2, 2½, 3, 3½, 4, 4½, 5, 5½, 6, 6½, 7 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of antibody to CCHFV which is administered to a patient prior to the administration of the very next dose in the sequence with no intervening doses.

The methods according to this aspect of the invention may comprise administering to a patient any number of secondary and/or tertiary doses of an antibody to CCHFV. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

Accordingly, in certain embodiments are provided pharmaceutical compositions comprising: one or more of the inventive antibodies or antigen-binding fragments thereof disclosed herein and throughout and a pharmaceutically acceptable carrier and/or one or more excipients. In certain other embodiments are provided pharmaceutical compositions comprising: one or more nucleic acid sequences encoding one or more inventive antibodies or antigen-binding fragments thereof; or one or more the expression vectors harboring such nucleic acid sequences; and a pharmaceutically acceptable carrier and/or one or more excipients.

### Therapeutic Uses of the Antibodies

Due to their binding to and interaction with CCHFV, it is believed that the inventive antibodies and antigen-binding fragments thereof are useful—without wishing to be bound to any theory—for preventing fusion of the virus with the host cell membrane, for preventing cell to cell virus spread, and for inhibition of syncytia formation. Alternatively, the antibodies of the present invention may be useful for ameliorating at least one symptom associated with the infection, such as fever, diarrhea, and bleeding, or for lessening the severity, duration, and/or frequency of the infection. The antibodies of the invention are also contemplated for prophylactic use in patients at risk for developing or acquiring a CCHFV infection. It is contemplated that the antibodies of the invention may be used alone, or in conjunction with a second agent, or third agent for treating CCHFV infection, or for alleviating at least one symptom or complication associated with the CCHFV infection, such as fever, diarrhea, or bleeding associated with, or resulting from such an infection. The second or third agents may be delivered concurrently with the antibodies of the invention, or they may be administered separately, either before or after the antibodies of the invention. The second or third agent may be an anti-viral such as ribavirin, an NSAID or other agents to reduce fever or pain, another second but different antibody that specifically binds CCHFV, an agent (e.g., an antibody) that binds to another CCHFV antigen, a vaccine against CCHFV, and an siRNA specific for a CCHFV antigen.

In yet a further embodiment of the invention the present antibodies are used for the preparation of a pharmaceutical composition for treating patients suffering from a CCHFV infection. In yet another embodiment of the invention the present antibodies are used for the preparation of a pharmaceutical composition for reducing the severity of a primary infection with CCHFV, or for reducing the duration of the infection, or for reducing at least one symptom associated with the CCHFV infection. In a further embodiment of the invention the present antibodies are used as adjunct therapy with any other agent useful for treating an CCHFV infection, including an antiviral, a toxoid, a vaccine, a second CCHFV antibody, or any other antibody specific for a CCHFV antigen, or any other palliative therapy known to those skilled in the art.

Accordingly, in certain embodiments are provided methods of treating or preventing a CCHFV infection, or at least one symptom associated with CCHFV infection, comprising administering to a patient in need thereof or suspected of being in need thereof one or more of the inventive antibodies or antigen-binding fragments thereof disclosed herein and throughout, such as, e.g., one or more of the anti-CCHFV antibodies disclosed in Table 3, such that the CCHFV infection is treated or prevented, or the at least one symptom associated with CCHFV infection is treated, alleviated, or reduced in severity.

In certain other embodiments are provided methods of treating or preventing a CCHFV infection, or at least one symptom associated with CCHFV infection, comprising administering to a patient in need thereof or suspected of being in need thereof a nucleic acid sequence encoding one or more of the inventive antibodies or antigen-binding fragments thereof, such nucleic acid sequenced disclosed in Table 3 and compliments thereof, such that the CCHFV infection is treated or prevented, or the at least one symptom associated with CCHFV infection is treated, alleviated, or reduced in severity.

In additional embodiments are provided methods of treating or preventing a CCHFV infection, or at least one symptom associated with CCHFV infection, comprising administering to a patient in need thereof or suspected of being in need thereof a host cell harboring a nucleic acid sequence or an expression vector comprising such a nucleic acid sequence, wherein such nucleic acid sequences is selected from the group consisting of sequences disclosed in Table 3 and compliments thereof, such that the CCHFV infection is treated or prevented, or the at least one symptom associated with CCHFV infection is treated, alleviated, or reduced in severity.

In additional embodiments are provided methods of treating or preventing a CCHFV infection, or at least one symptom associated with CCHFV infection, comprising administering to a patient in need thereof or suspected of being in need thereof a pharmaceutical composition comprising either: one or more of the inventive antibodies or antigen-binding fragments thereof as disclosed in Table 3; one or more nucleic acid sequences or an expression vectors comprising such a nucleic acid sequence, wherein such nucleic acid sequences are selected from the group consisting of sequences disclosed in Table 3 and compliments thereof; one or more host cells harboring one or more nucleic acid sequences or an expression vectors comprising such one or more nucleic acid sequences, wherein such nucleic acid sequences are selected from the group consisting of sequences disclosed in Table 3 and compliments thereof; and a pharmaceutically acceptable carrier and/or one or more excipients, such that the CCHFV infection is treated or prevented, or the at least one symptom associated with CCHFV infection is treated, alleviated, or reduced in severity.

In certain embodiments are provided methods of treating or preventing a CCHFV infection, or at least one symptom associated with said CCHFV infection, comprising administering to a patient in need thereof or suspected of being in need thereof one or more of the inventive antibodies or antigen-binding fragments thereof disclosed herein and throughout, such as, e.g., one or more of the anti-CCHFV antibodies disclosed in Table 3, such that the CCHFV infection is treated or prevented, or the at least one symptom associated with CCHFV infection is treated, alleviated, or reduced in severity.

In certain other embodiments are provided methods of treating or preventing a CCHFV infection, or at least one symptom associated with said CCHFV infection, comprising administering to a patient in need thereof or suspected of being in need thereof a nucleic acid sequence encoding one or more of the inventive antibodies or antigen-binding fragments thereof, such nucleic acid sequenced disclosed in Table 3 and compliments thereof, such that the CCHFV infection is treated or prevented, or the at least one symptom associated with CCHFV infection is treated, alleviated, or reduced in severity.

In additional embodiments are provided methods of treating or preventing a CCHFV infection, or at least one symptom associated with said CCHFV infection, comprising administering to a patient in need thereof or suspected of being in need thereof a host cell harboring a nucleic acid sequence or an expression vector comprising such a nucleic acid sequence, wherein such nucleic acid sequences is selected from the group consisting of sequences disclosed in Table 3 and compliments thereof, such that the CCHFV infection is treated or prevented, or the at least one symptom associated with CCHFV infection is treated, alleviated, or reduced in severity.

In additional embodiments are provided methods of treating or preventing a CCHFV infection, or at least one symptom associated with said CCHFV infection, comprising administering to a patient in need thereof or suspected of being in need thereof a pharmaceutical composition comprising either: one or more of the inventive antibodies or antigen-binding fragments thereof as disclosed in Table 3; one or more nucleic acid sequences or an expression vectors comprising such a nucleic acid sequence, wherein such nucleic acid sequences are selected from the group consisting of sequences disclosed in Table 3 and compliments thereof; one or more host cells harboring one or more nucleic acid sequences or an expression vectors comprising such one or more nucleic acid sequences, wherein such nucleic acid sequences are selected from the group consisting of sequences disclosed in Table 3 and compliments thereof; and a pharmaceutically acceptable carrier and/or one or more excipients, such that the CCHFV infection is treated or prevented, or the at least one symptom associated with CCHFV infection is treated, alleviated, or reduced in severity.

### Combination Therapies

As noted above, according to certain embodiments, the disclosed methods comprise administering to the subject one or more additional therapeutic agents in combination with an antibody to CCHFV. As used herein, the expression “in combination with” means that the additional therapeutic agents are administered before, after, or concurrent with the pharmaceutical composition comprising the anti-CCHFV antibody. The term “in combination with” also includes sequential or concomitant administration of the anti-CCHFV antibody and a second therapeutic agent.

For example, when administered “before” the pharmaceutical composition comprising the anti-CCHFV antibody, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the anti-CCHFV antibody. When administered “after” the pharmaceutical composition comprising the anti-CCHFV antibody, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the anti-CCHFV antibodies. Administration “concurrent” or with the pharmaceutical composition comprising the anti-CCHFV antibody means that the additional therapeutic agent is administered to the subject in a separate dosage form within less than 5 minutes (before, after, or at the same time) of administration of the pharmaceutical composition comprising the anti-CCHFV antibody, or administered to the subject as a single combined dosage formulation comprising both the additional therapeutic agent and the anti-CCHFV antibody.

Combination therapies may include an anti-CCHFV antibody of the invention and any additional therapeutic agent that may be advantageously combined with an antibody of the invention, or with a biologically active fragment of an antibody of the invention.

For example, a second or third therapeutic agent may be employed to aid in reducing the viral load in the lungs, such as an antiviral, for example, ribavirin. The antibodies may also be used in conjunction with other therapies, as noted above, including a toxoid, a vaccine specific for CCHFV, a second antibody specific for CCHFV, or an antibody specific for another CCHFV antigen.

### Diagnostic Uses of the Antibodies

The inventive anti-CCHFV antibodies and antigen-binding fragments thereof may also be used to detect and/or measure CCHFV in a sample, e.g., for diagnostic purposes. It is envisioned that confirmation of an infection thought to be caused by CCHFV may be made by measuring the presence of the virus through use of any one or more of the antibodies of the invention. Exemplary diagnostic assays for CCHFV may comprise, e.g., contacting a sample, obtained from a patient, with an anti-CCHFV antibody of the invention, wherein the CCHFV antibody is labeled with a detectable label or reporter molecule or used as a capture ligand to selectively isolate the virus containing the protein from patient samples. Alternatively, an unlabeled CCHFV antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure CCHFV in a sample include enzyme-linked immunosorbent assay (ELISA), radioimmunoassay (MA), and fluorescence-activated cell sorting (FACS).

Samples that can be used in CCHFV diagnostic assays according to the present invention include any tissue or fluid sample obtainable from a patient, which contains detectable quantities of CCHFV protein, or fragments thereof, under normal or pathological conditions. Generally, levels of CCHFV in a particular sample obtained from a healthy patient (e.g., a patient not afflicted with a disease or condition associated with the presence of CCHFV) will be measured to initially establish a baseline, or standard, level of CCHFV protein. This baseline level of CCHFV can then be compared against the levels of CCHFV measured in samples obtained from individuals suspected of having an CCHFV infection, or symptoms associated with such infection.

### EXAMPLES

Applicants have comprehensively profiled the human antibody response to CCHFV by isolating and characterizing 393 CCHFV-specific monoclonal antibodies from the memory B cells of four CCHFV-convalescent donors and used these antibodies to comprehensively map the antigenic topology of CCHFV. The antibody response to CCHFV was determined to be comprised of a number of clones that target several antigenic sites. The majority of antibodies target the Gc subunit and are cross-reactive for multiple GnGc strains, providing strong support for the development of CCHFV antibodies that target the Gn and/or Gc subunits. Taken together, the results have implications for the design and evaluation of CCHFV vaccine and antibody-based therapeutic candidates and offer new options for passive prophylaxis.

### Generation of CCHFV Sorting Probes

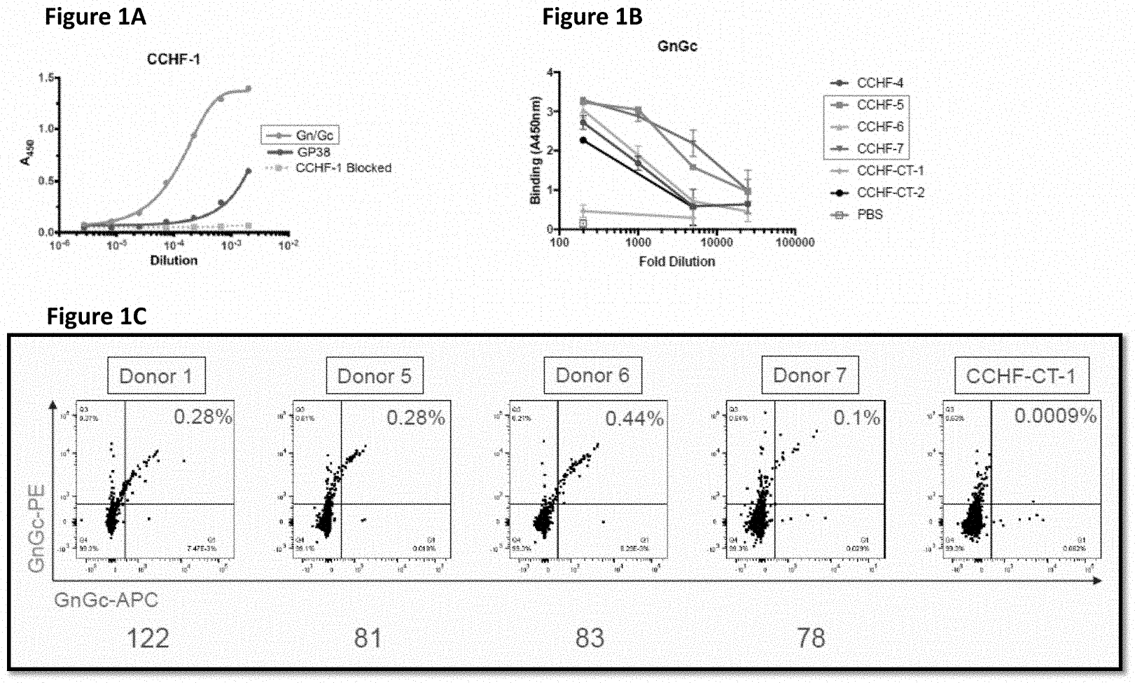

The CCHFV GnGc and GP38 glycoproteins were engineered (transmembrane regions deleted) to be secreted as soluble recombinantly expressed proteins from Schneider 2 insect cells. The proteins were then purified using standard Strep II tag purification techiniques. The purified proteins were then complexed with labled streptavidin probes (utilizing the Strep-II tags fused to the proteins) together providing the antigen specific probes required to support single cell fluorescence activated B-cell sorting and resulting antibody isolation from CCHFV-convalescent adult human donors (see FIG. 1A).

Isolation of CCHFV-Specific Monoclonal Antibodies from CCHFV-Convalescent Adult Human Donors

In order to comprehensively profile the human antibody response to CCHFV, approximately 364 monoclonal antibodies from the memory B cells of four CCHFV-convalescent adult donors (“Donor 1”, “Donor 5”, “Donor 6”, and “Donor 7”) were isolated and characterized. 450 GnGc-specific B cells were single-cell sorted from a Donor 1 sample, and 184 GnGc-specific B cells were sorted for samples from Donor 5, Donor 6, and Donor 7. Antibody variable heavy (VH) and variable light (VL) chain genes were rescued by single-cell PCR. Tiller et al. (2008) J Immunol Methods 329, 112-124. Cognate heavy and light chain pairs were subsequently cloned and expressed as full-length IgGs in an engineered strain of Saccharomyces cerevisiae for further characterization. Bornholdt et al., (2016) Science 351, 1078-1083.

The ability of serum from CCHFV-convalescent donors to bind to the GnGc probe was tested. B cells isolated from the donors bind to GnGc (see FIG. 1B). GnGc reactivity of B cells from each of the CCHF-convalescent donors was demonstrated via FACS (see FIG. 1C).

### B Cell Classification and Sequence Analysis of CCHFV-Specific Antibody Repertoires

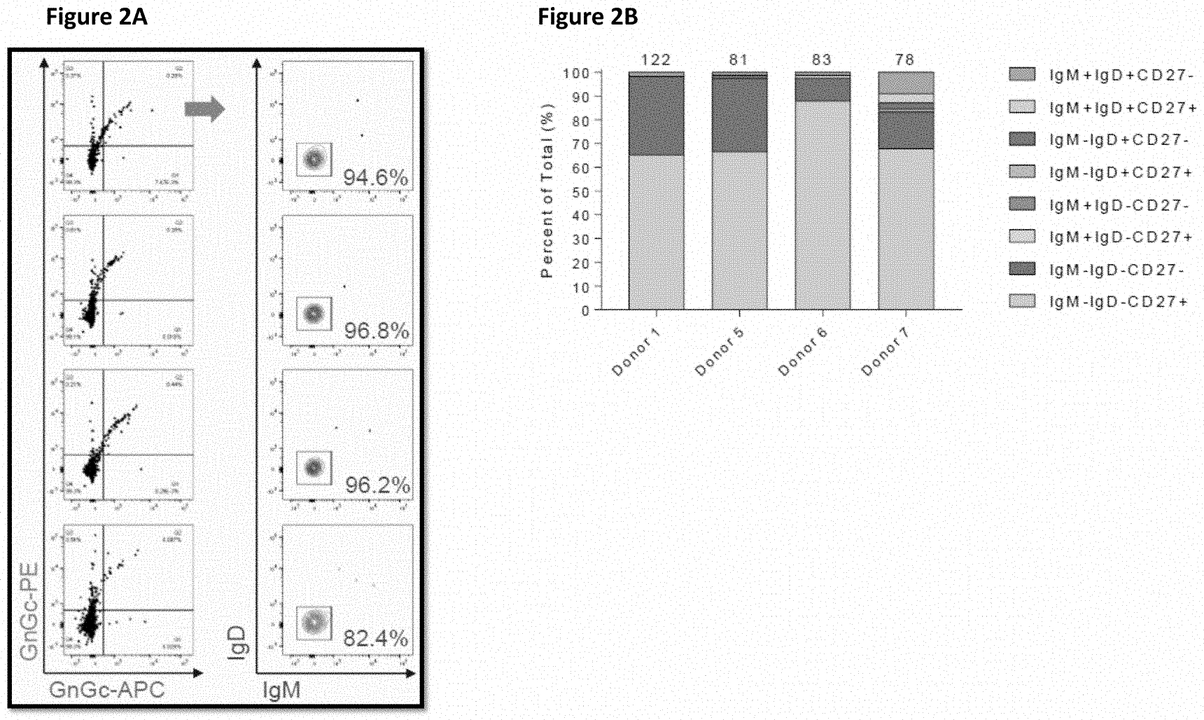

The types and proportions of B cell subsets present were examined. IgD and IgM staining profiles reveal that the CCHFV-specific repertoires are class-switched, meaning that the cells are affinity matured (see FIG. 2A). Consistent with this observation, the majority of B cells show an IgM-IgD-CD27+phenotype (see FIG. 2B). Thus, most antibodies were derived from classical memory B cells.

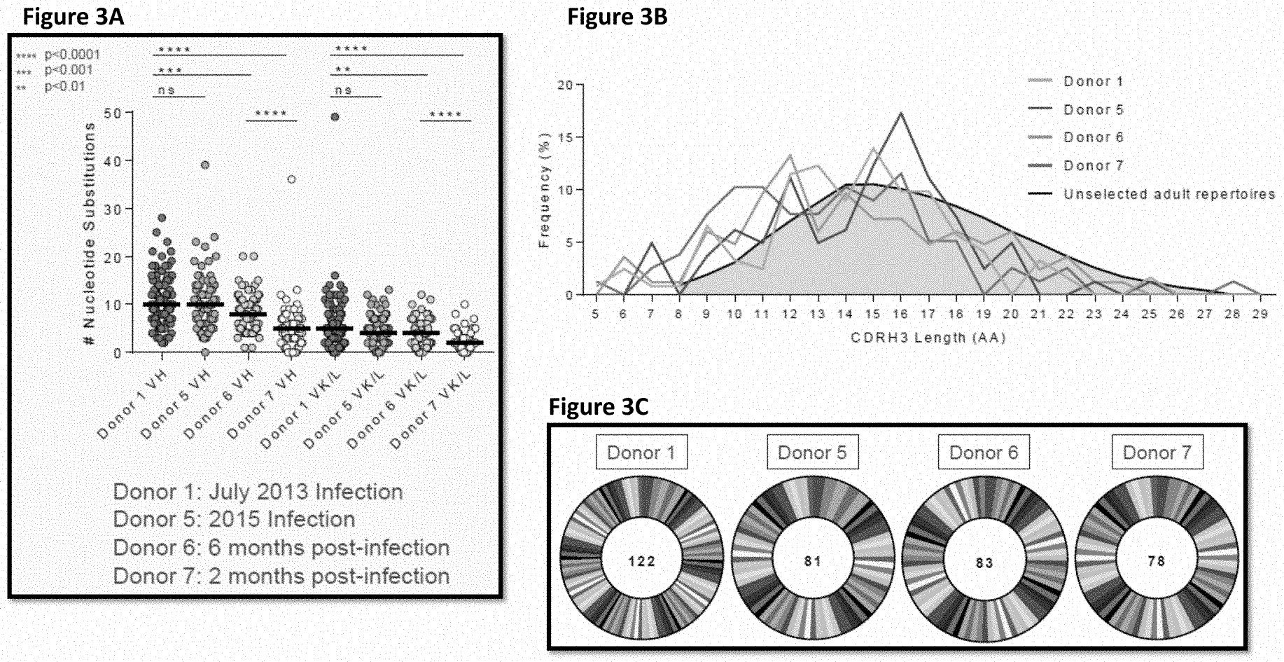

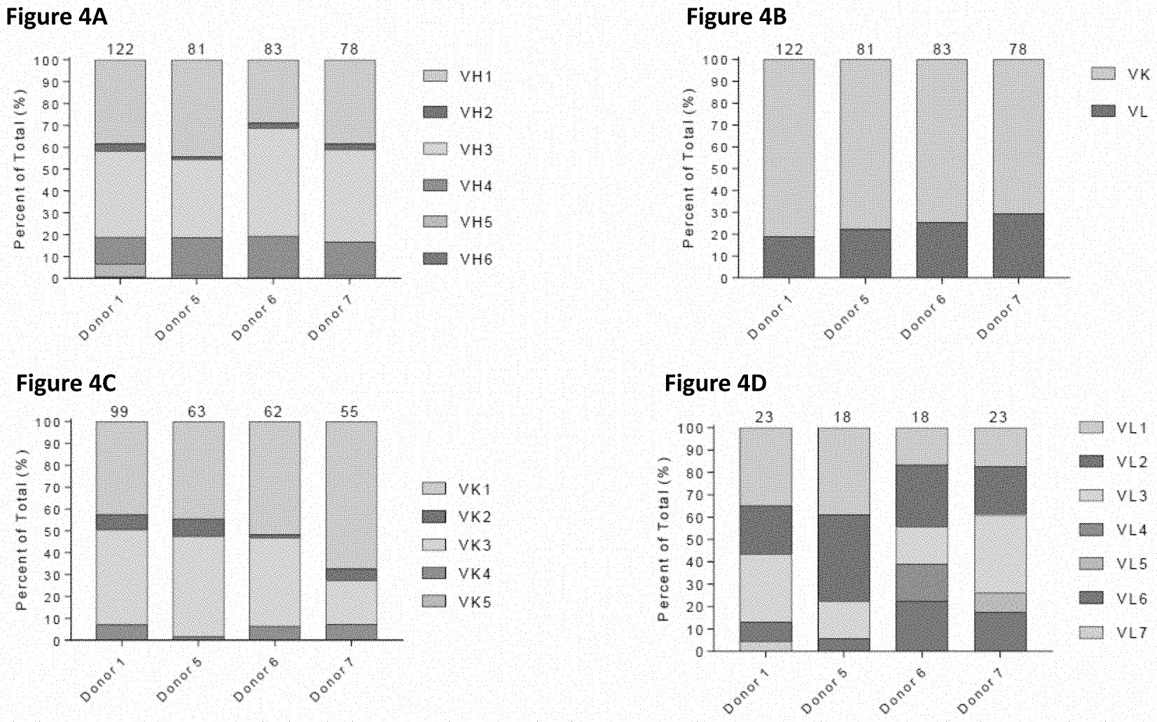

Sequence analysis of isolated monoclonal antibodies revealed that the CCHFV-specific repertoire is highly diverse, with few to no expanded clonal lineages. More than 70 unique lineages were observed for each donor (see FIG. 3C). CDRH3 lengths are similar to unselected repertoires (generally, approximately 13-17 amino acids in length; FIG. 3B). The average level of somatic hypermutation (SHM) ranged between 0 and 30 nucleotide substitutions per VH gene (excluding CDRH3) and 0 and 12 substitutions per VK/L gene (see FIG. 3A). Somatic mutation load may correlate with time post-infection, as the number of nucleotide substitutions in Donors 6 and 7 (two months post-infection) is significantly less than the number of nucleotide substitutions in Donors 1 and 5. Sequence analysis also revealed that CCHFV-specific repertoires tend to use similar proportions of VH, VK, and VL families. For example, the repertoires tend to use VH1 or VH3 (see FIG. 4A) and tend to use VK rather than VL (see FIG. 4B). If VK is chosen, the repertoires tend to use VK1 and VK3 (FIG. 4C); if V_{L} is chosen, the repertoires tend to use VL1, VL2, and VL3 (see FIG. 4D).

### Antibody Binding Studies

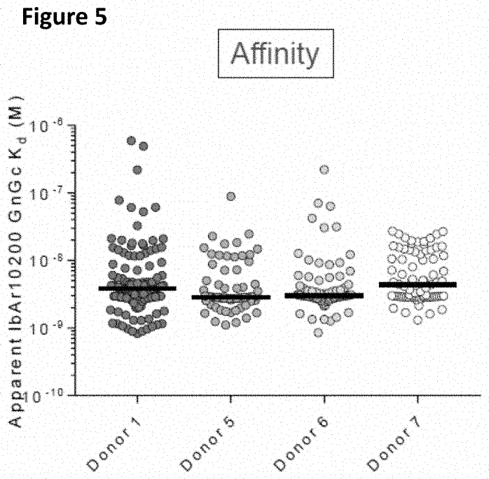

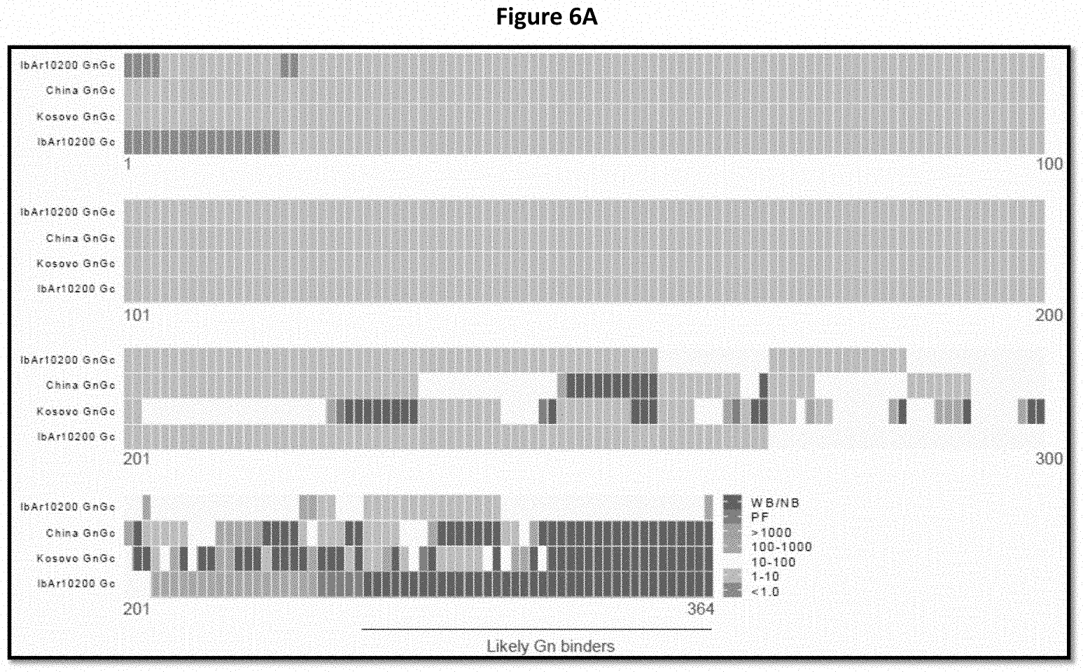

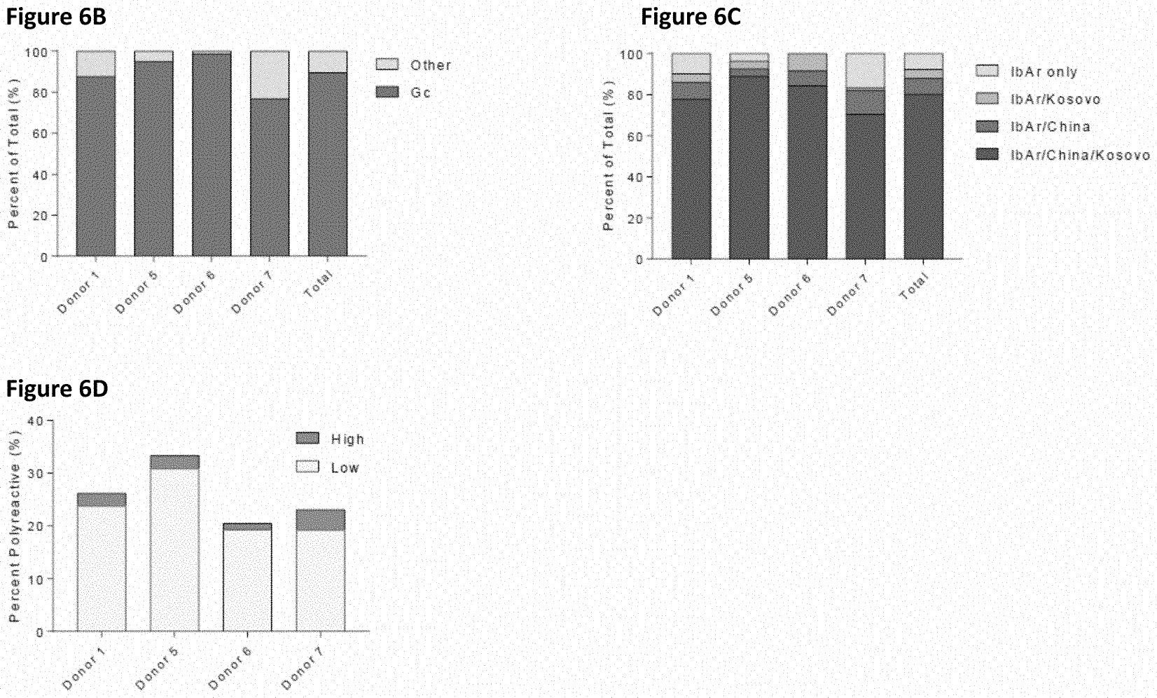

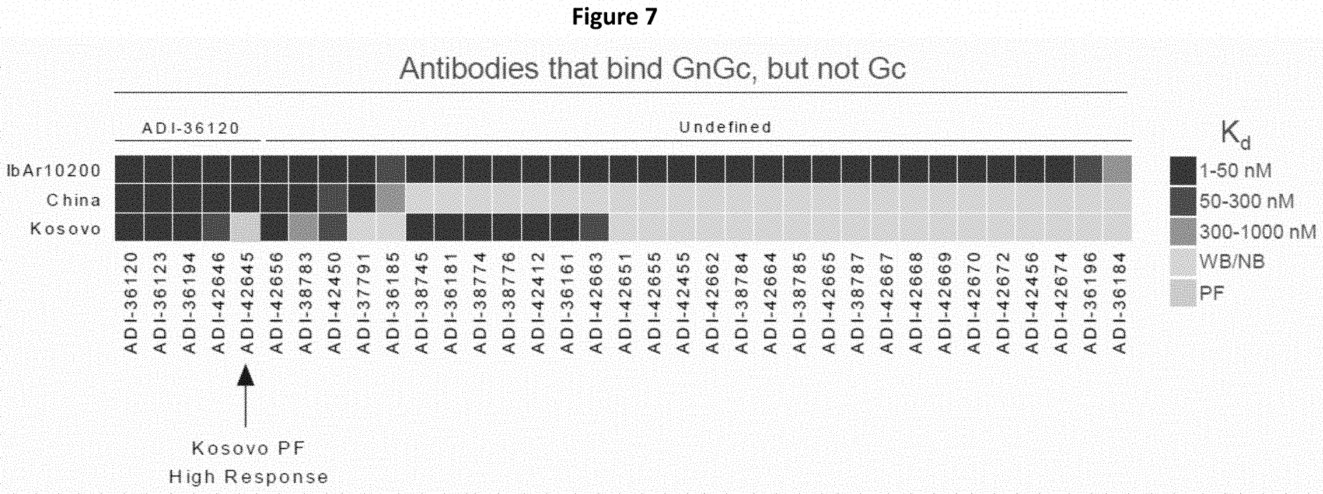

The apparent binding affinities of the IgGs for IbAr10200 GnGc were measured using biolayer interferometry. McLellan et al., (2011) J Virol 85, 7788-7796 (2011). IbAr10200 is a CCHF virus strain originally isolated from Hyalomma excavatum ticks from Sokoto, Nigeria in 1966. Results show that antibodies from each donor bind with high affinity to IbAr10200 GnGc (see FIG. 5 and Table 2). Interestingly, a relatively large proportion of the antibodies not only bound to IbAr10200 GnGc but also other strains of CCHFV—China GnGc and Kosovo GnGc—as well as just the Gc subunit (IbAr10200 Gc) (FIGS. 6A and 6C). More than 70% of the antibodies isolated from each donor bound to Gc (FIG. 6B). FIG. 7 shows that antibodies in some bins, such as the ADI-36120 bin, bound to all CCHFV strains with high affinity. Conversely, antibodies from an undefined bin bound only one or two of the CCHFV strains. Such results suggest that the antibodies of the ADI-36120 bin may bind a conserved Gn or quaternary epitope.

Since certain antiviral antibody specificities have been associated with poly- and autoreactivity, the CCHFV antibodies were tested for polyreactivity using a previously described high-throughput assay that correlates with down-stream behaviors such as serum clearance. Andrews et al. (2015), Sci Transl Med 7, 316ra192; Kelly et al. (2015), MAbs, 0; Xu et al. (2013), Protein Eng Des Sel 26, 663-670. The vast majority of CCHFV antibodies lack significant polyreactivity in this assay (see FIG. 6D).

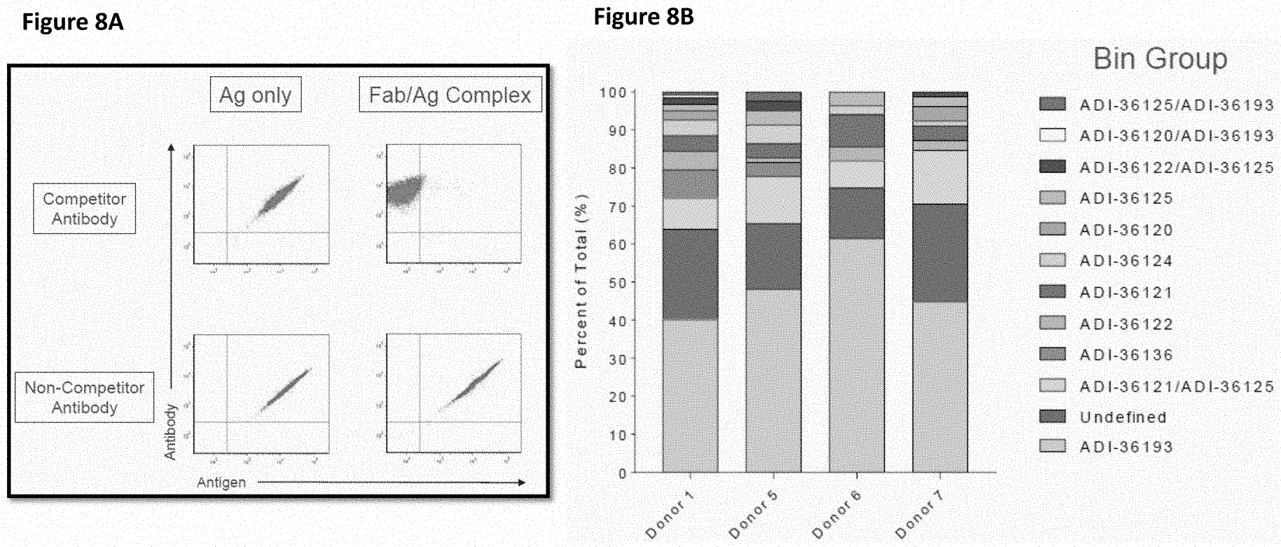

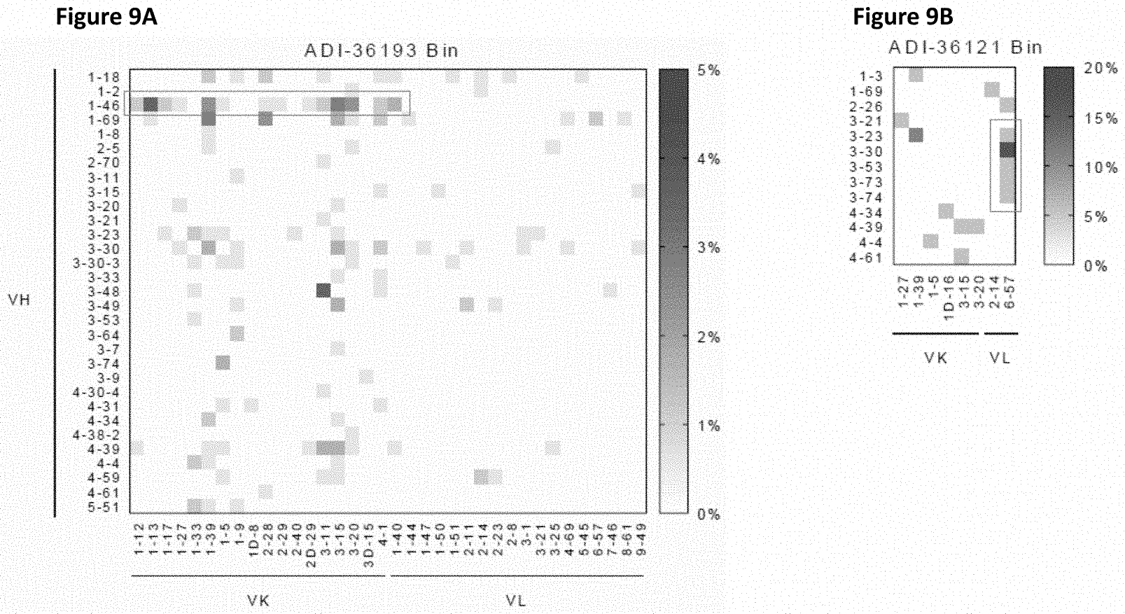

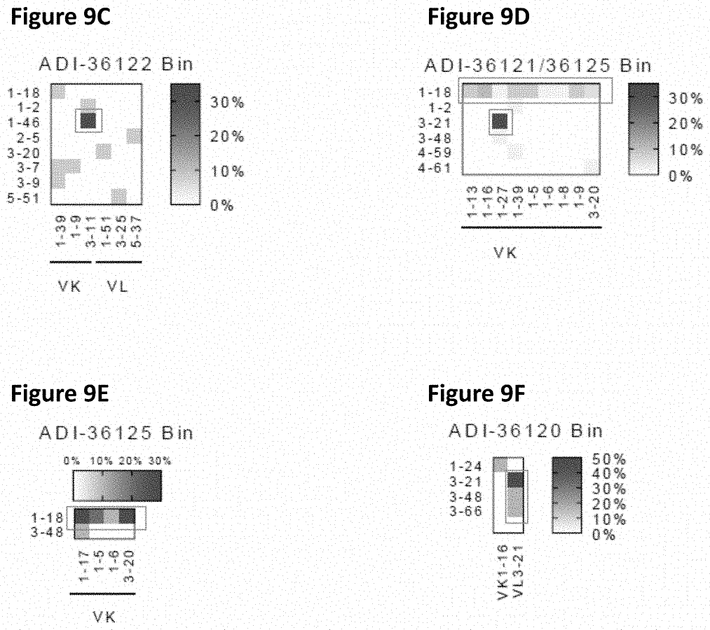

To further analyze the binding affinities of the CCHFV antibodies, antibodies were sorted by bin and performed competitive binding experiments using a previously described yeast-based assay. Bowley et al. (2007), Protein Eng Des Sel 20, 81-90. Epitope binning of the antibodies was performed on a Forte Bio Octet Red384 system (Pall Forte Bio Corporation, Menlo Park, Calif.) using a standard sandwich format binning assay. No binding following addition of a Fab/Ag complex indicates epitope blocking (competitor), while binding after addition of the Fab/Ag complex indicates an unoccupied epitope (non-competitor) (see FIG. 8A). The majority of antibodies from each donor bin with ADI-36193 (see FIG. 8B). Further, a significant portion of antibodies from each donor were not binned. Sequence analysis of antibodies in several bins revealed germline preferences. For example, within the ADI-36193 bin, antibodies showed preferences for VH1-46 (see FIG. 9A). Other germline preferences are highlighted in FIGS. 9B-9F and in Table 1.

Highly Potent Neutralizing Antibodies from Bins ADI-36193 and ADI-36121

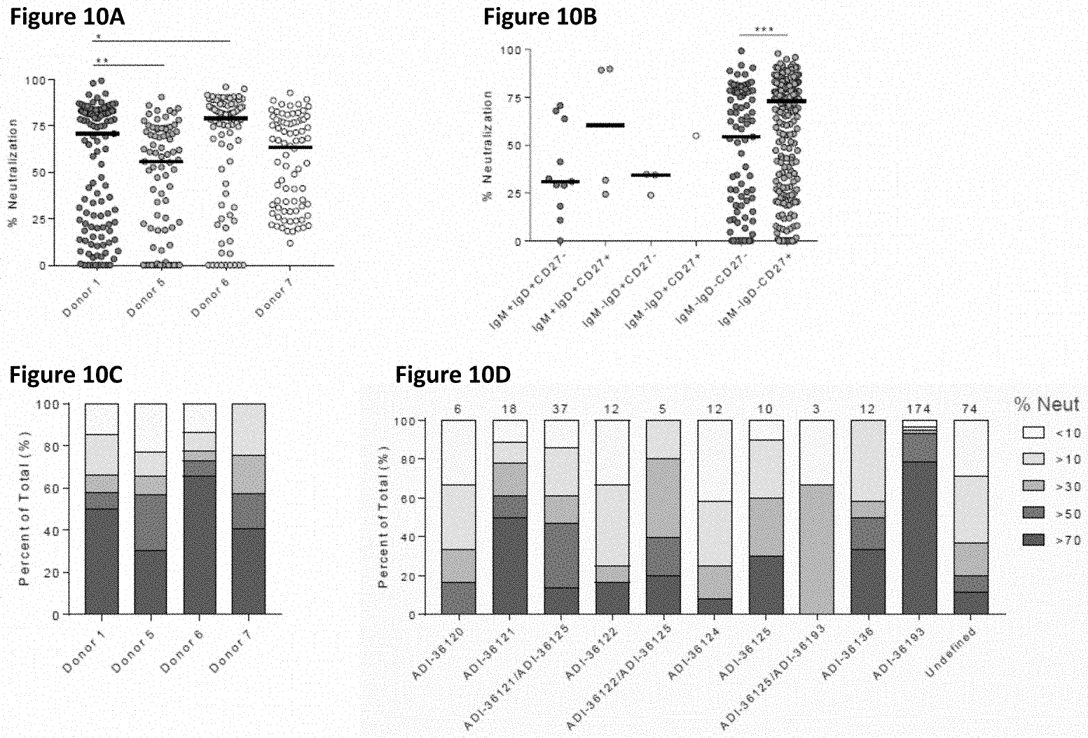

The antibodies were next tested for neutralizing activity using a previously described high-throughput neutralization assay. McLellan et al. (2013), Science 342, 592-598. Greater than 50% of the isolated antibodies showed neutralizing activity (FIG. 10A), with highest neutralizing activity observed in the IgM-IgD-CD27+subtype (see FIG. 10B). More than half of antibodies from each donor showed at least 50 percent neutralization or greater (see FIG. 10C and Table 2). Antibodies from bins ADI-36193 and ADI-36121 showed the highest proportion of neutralizing antibodies (see FIG. 10D).

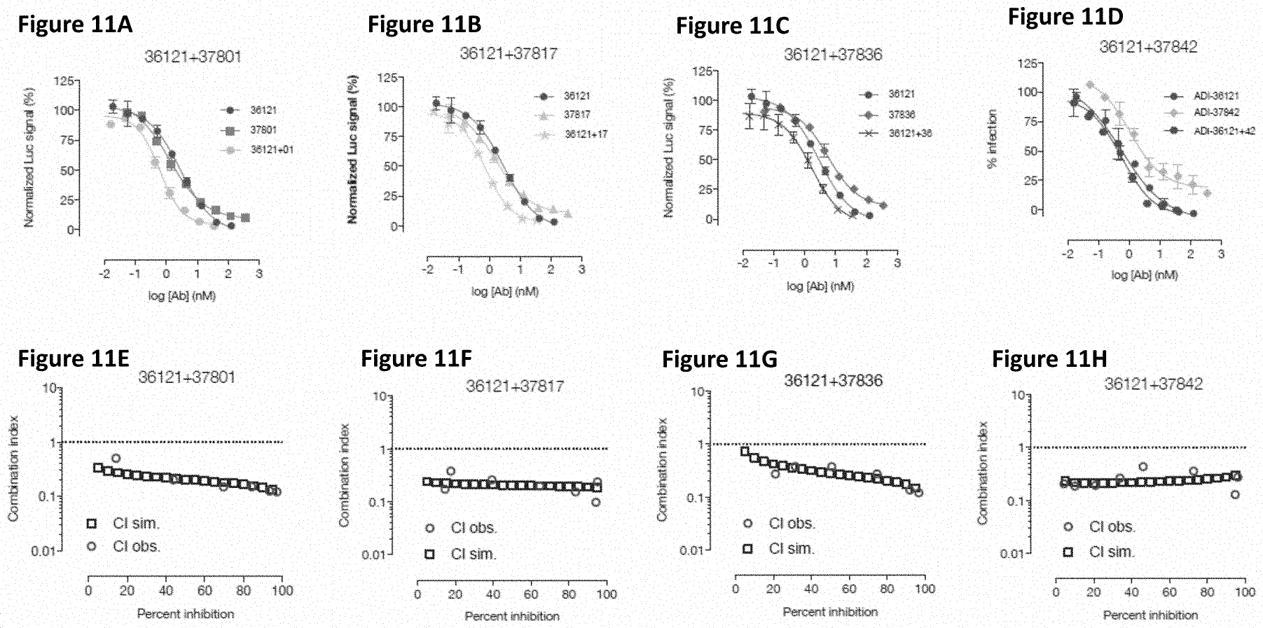

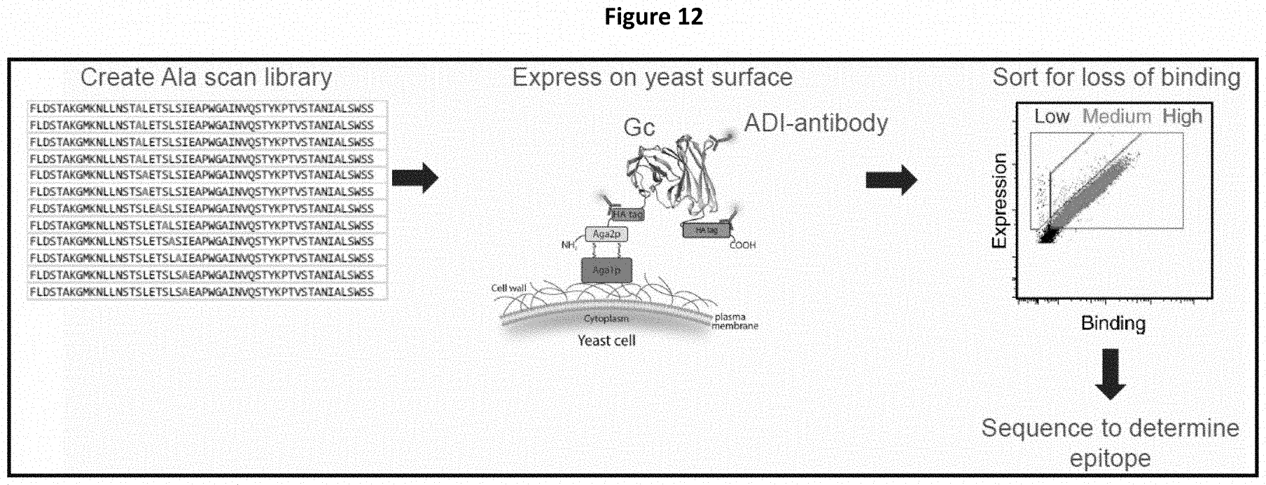

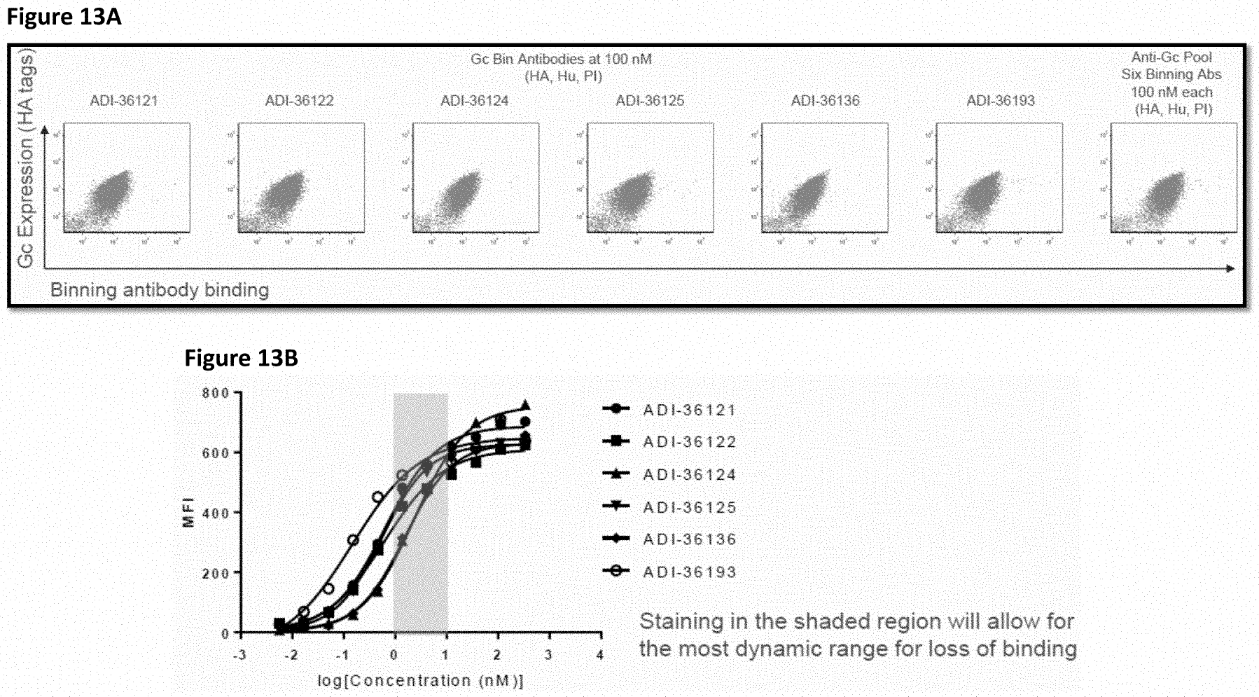

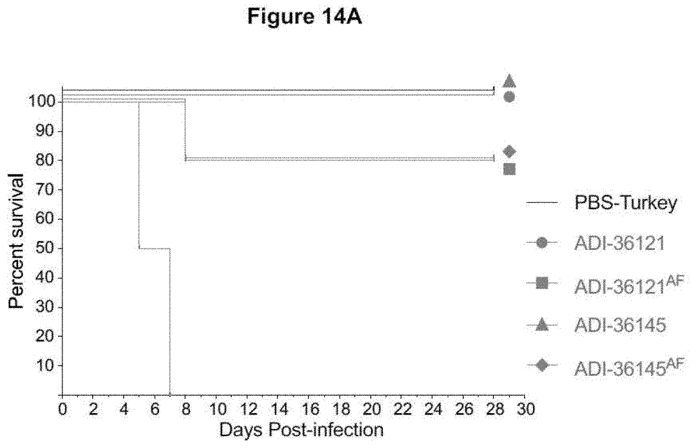

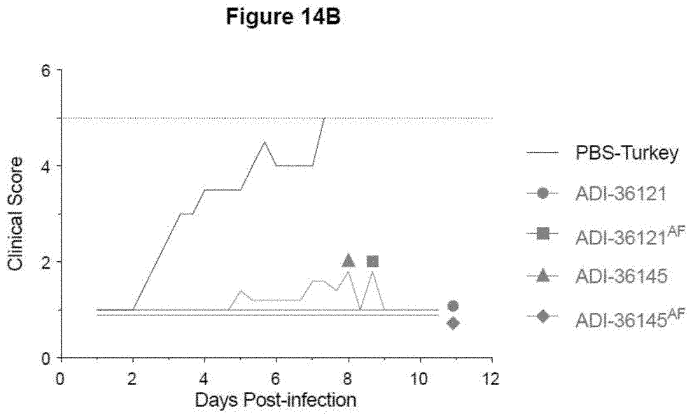

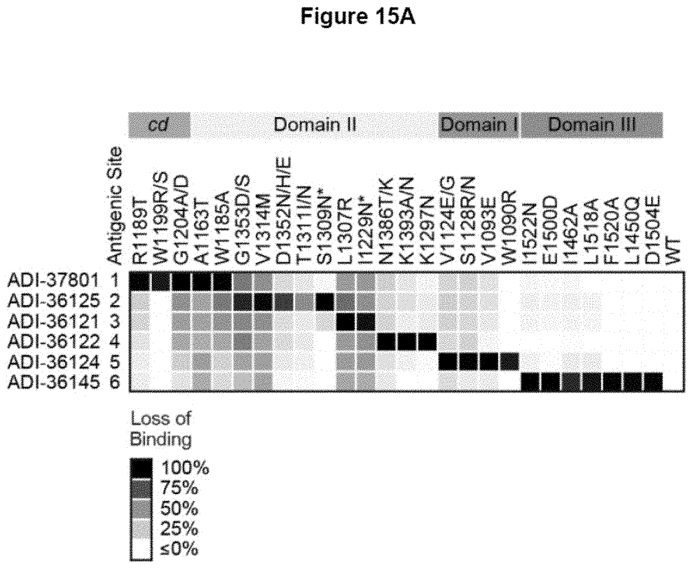

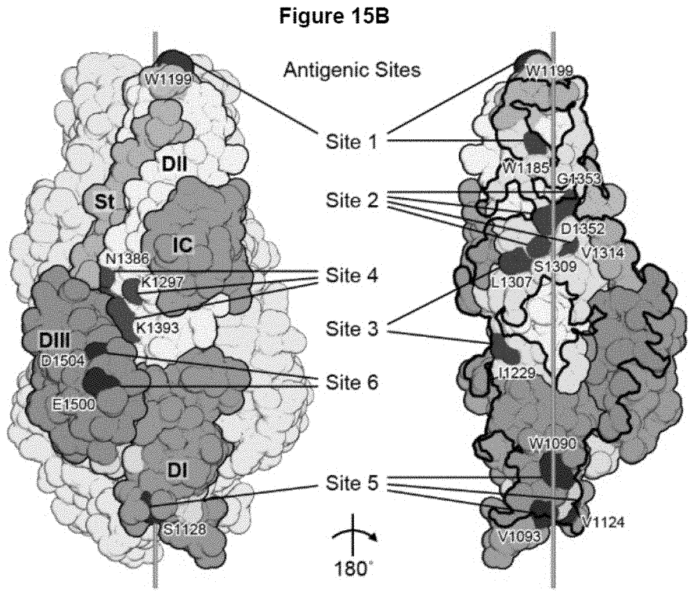

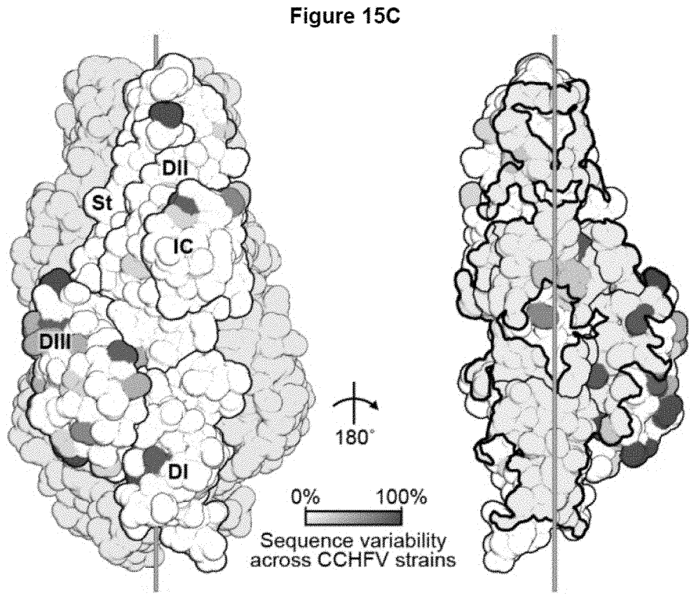

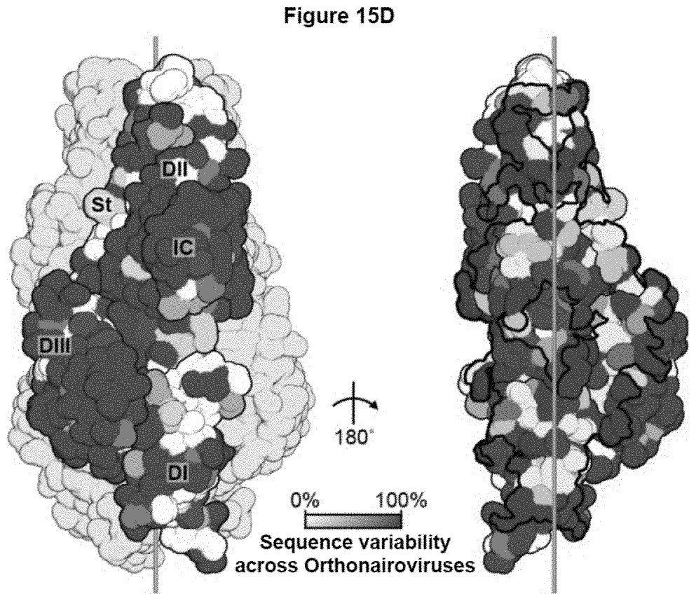

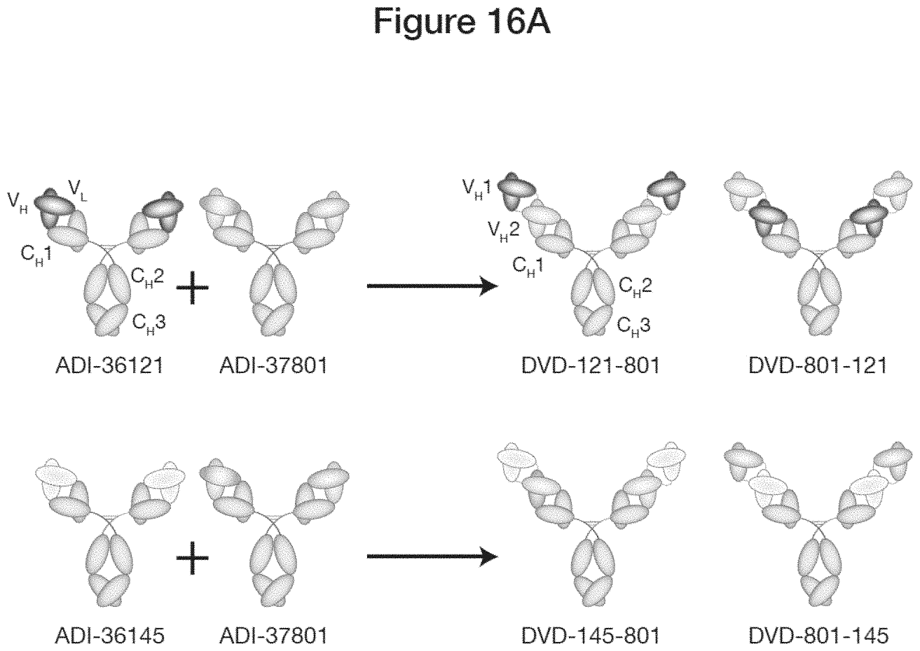

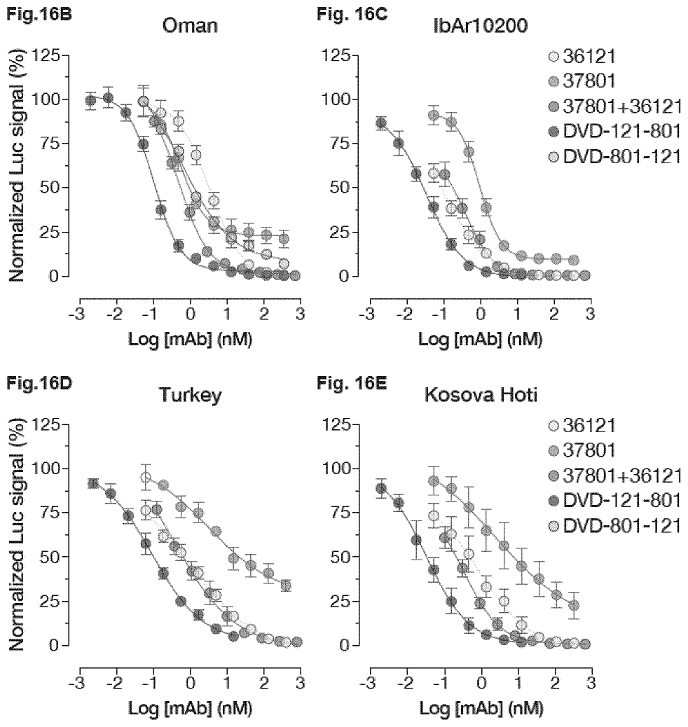

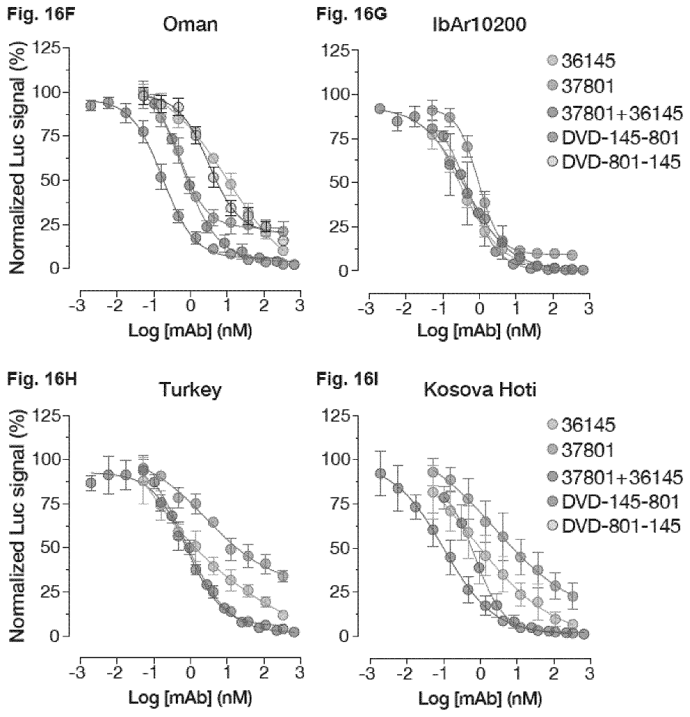

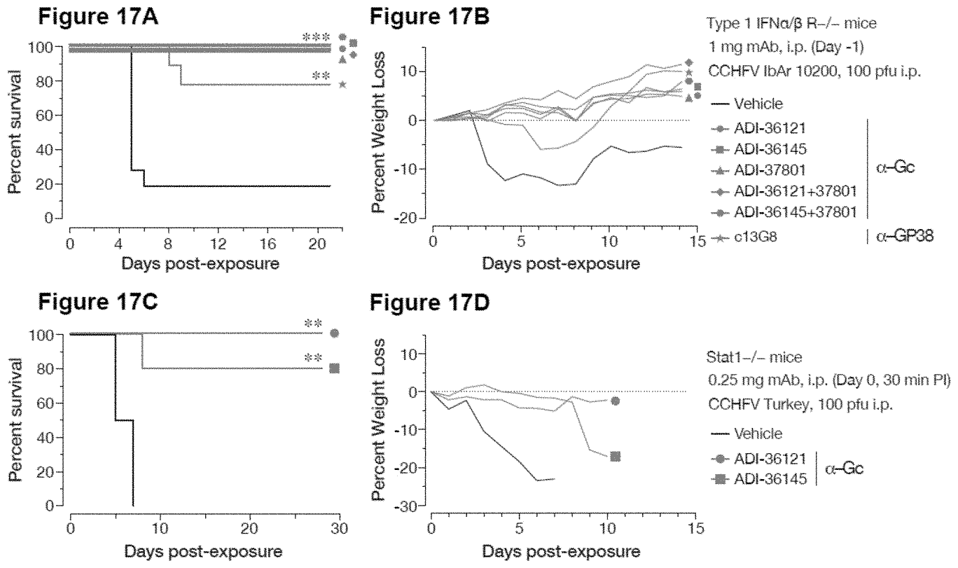

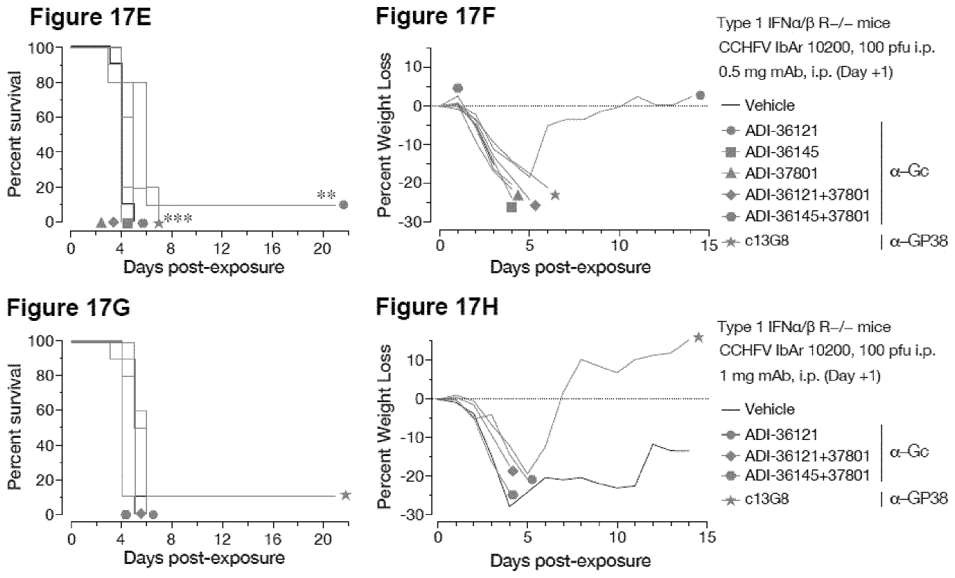

FIG. 11A through FIG. 11D demonstrate that ADI-36121 potently neutralized CCHF virus-like particles (VLPs) by itself, while ADI-36193 bin members leave an unneutralized fraction. Combination index analysis of ADI-36121 in combination with ADI-36193 bin members showed CI values of less than 1, indicating synergistic inhibition of CCHFV VLPs for both observed data and data fitted through linear regression. Such neutralization is at a higher potency than either component alone.

### Epitope Mapping Studies

To determine where and/or how the antibodies of the present disclosure bind CCHFV, additional experiments directed at binding may be conducted. FIG. 12 illustrates a methodology for epitope mapping for anti-Gc antibodies. The methodology may include creating an alanine (ala) scanning library, expressing the Gc proteins on a yeast surface, sorting for loss of binding, and sequencing to determine epitope. Gc has previously been shown to be expressed conformationally intact on the surface of yeast. FIG. 13A demonstrates that all anti-Gc binning antibodies bind to surfaced displayed Gc. By using antibody concentrations highlighted in FIG. 13B, the most dynamic range for loss of binding can be observed, and thus epitope can be determined.

### Protective Efficacy of ADI-36121 and ADI-36145.

FIG. 14A shows survival curves for mice challenged with Turkey2004 and treated with a single 250 μg dose of the indicated mAb 30 minutes post-infection. **, Mantel-Cox P<0.01. FIG. 14B are clinical scores of animals within the study cohort are shown. While there are no significant differences in the protective efficacy between ADI-36121 and ADI-36145, ADI-36121 appeared to show greater clinical benefit than ADI-36145 through the course of the study.

FIG. 15A through FIG. 15D. Mapping of antigenic sites and surface conservation of CCHFV Gc. A) Heat map depicting the magnitude of loss of binding of single mutations compared to wild type. Darker shade indicates a greater loss of binding. For residues with multiple mutations, averages of binding loss for a mutation at a given position are shown. * denotes the introduction of a potential N-linked glycosylation site. B) Antigenic sites mapped on the surface of one CCHFV Gc protomer within the post-fusion trimer. The trimer axis is shown in light blue. Only the front Gc subunit is shown in the right panel, after a 180 degrees rotation about the trimer axis. The trimer interface is outlined in black. All residues from panel A that are not highlighted in panel B are occluded from the surface. C) Sequence similarity across 15 representative CCHFV strains color-plotted on the Gc surface. D) Sequence similarity across 14 different orthonairoviruses. IC indicates the nairovirus-specific “insertions cluster” displayed in panel B.

FIG. 16A through FIG. 16I. Neutralization activity and protective efficacy of engineered bsAbs. (FIG. 16A) Schematic illustration of candidate nAb (or “neutralizing antibody”) IgGls and dual-variable domain IgGs (DVD-Igs) derived from them by combining IgG1 variable domains. (FIG. 16B through FIG. 16I) Neutralization curves of the indicated nAbs, nAb combinations, and bsAbs against against tecVLPs bearing Gn/Gc proteins from (FIG. 16B, FIG. 16F) Oman, (FIG. 16C, FIG. 16G) IbAr10200, (FIG. 16D, FIG. 16H) Turkey, (FIG. 16E, FIG. 16I) Kosova Hoti strains.

FIG. 17A through FIG. 17H. Protective efficacy of lead nAbs and nAb combinations in two murine models of lethal CCHFV challenge. (FIG. 17A, FIG. 17B) Stat1−/− mice were challenged with CCHFV Turkey/2004 and then treated with single doses of the indicated mAbs or vehicle at 30 min post-exposure. (n=5 mice per group) (FIG. 17A) Survival curves (vehicle versus test mAb) were compared by Mantel-Cox test (*** P<0.001, ** P<0.01). (FIG. 17B) Associated mean weight loss data are shown. (FIG. 17C, FIG. 17D) Type I IFN α/β/R−/− (IFNAR1-KO) mice were treated with the indicated mAbs or mAb combinations 1 day prior to challenge with CCHFV IbAr10200. (n=10 mice per group) (FIG. 17C) Survival curves (vehicle versus test mAb) were compared by Mantel-Cox test (*** P<0.001, ** P<0.01). (FIG. 17D) Associated mean weight loss data are shown. (FIG. 17E through FIG. 17H) IFNAR1-KO mice were exposed to CCHFV IbAr10200 and treated with the indicated mAbs or mAb combinations at 1 day post-challenge. (n=10 mice per group) (FIG. 17E, FIG. 17G) Survival curves (vehicle versus test mAb) were compared by Mantel-Cox test (*** P<0.001, ** P<0.01). (FIG. 17F, FIG. 17H) Associated mean weight loss data are shown.

### Discussion

An in-depth understanding of the human antibody response to CCHFV infection will aid the development and evaluation of CCHFV vaccine and therapeutic and/or prophylactic antibody candidates for the treatment and/or prevention of CCHFV infection. The specificities and functional properties of antibodies induced by natural CCHFV infection have remained largely undefined. As disclosed herein, a high-throughput antibody isolation platform and a collection of GnGc-specific B cells were used to dissect the human memory B cell response to CCHFV in four naturally infected adult donors, and highly potent and selective CCHFV-neutralizing antibodies were isolated and characterized.

To enhance neutralization potency and breadth and mitigate the risk of viral mutational escape (Gilchuk et al., Immunity 52, 388-402, 2020; Keeffe et al., Cell Rep. 25, 1385-1394, 2018; Wec et al., Science 354, 350-354, 2019), we evaluated combinations of non-competing nAbs. Only combinations of Site 3/domain II or Site 6/domain III binders with Site 1/fusion loop binders afforded synergistic neutralization, characterized by Chou-Talalay combination index (CI) scores <1 and improvements in both neutralization IC50 and un-neutralized fraction. The site-specificity of nAb synergy suggested that it did not arise solely from the simultaneous engagement of two non-overlapping functional epitopes (Diamant et al., 2015). Instead, we speculate that these synergies reflect cooperative binding. Specifically, the Site 3/Site 6 nAbs may trap “open” Gc conformers in which the fusion loops are more exposed for recognition by Site 1 nAbs. The further improvements in neutralization IC50 with bsAbs may reflect increases in binding avidity of the physically linked variable domains to these pairs of sites, which may be further magnified by the tetravalent DVD format used herein (Diamant et al., A Platform for Next-Generation Anti-Toxin Drugs: Toxins (Base1) 7, 1854-1881 2015; Jakob et al., MAbs 5, 358-363, 2013; Wec et al., Microbe 25, 39-48, 2016). The reduced potency of the alternate DVD-Ig configurations bearing the fusion loop-binding domains as outer domains (DVD-801-121 and DVD-801-145) presumably reflects structural constraints that reduce the efficiency of bivalent and/or tetravalent bsAb engagement with Gc. An understanding of these constraints awaits structural elucidation of the supramolecular organization of CCHFV Gn/Gc in intact viral particles.

Single doses of candidate nAbs targeting Gc domains II and III afforded prophylactic protection against virulent CCHFV strains from two distinct viral clades. That these studies were independently performed in two BSL-4 laboratories using genetically distinct immunocompromised murine models attests to the robustness of our findings. However, single doses of all tested nAbs, synergistic nAb combinations, and non-neutralizing GP38-specific mAbs failed to protect mice in a more stringent therapeutic setting. Strikingly, a single bsAb, DVD-121-801, combining variable domains from the synergizing nAbs ADI-36121 (Site 3/domain II) and ADI-37801 (Site 1/fusion loops), protected mice when administered 24 h post-viral challenge, concordant with its enhanced neutralization potency against tecVLPs bearing Gn/Gc from the cognate challenge strain. We speculate that this bsAb may also benefit from enhanced Fc-mediated effector functions by virtue of its increased length and/or flexibility relative to its IgG1 precursors. DVD-121-801 is the first antibody-based treatment demonstrated to afford therapeutic protection against lethal CCHFV challenge with a single dose, and it is a lead candidate for further evaluation in murine and nonhuman primate models of CCHFV challenge (Cross et al., PLoS Negl. Trop. Dis. 14, e0008637, 2020; Haddock et al., Nat. Microbiol. 3, 556-562, 2018).

The development of an effective CCHFV vaccine has presented a number of unique challenges, and selection of the optimal vaccination strategy will be of the utmost importance. The in-depth analysis of the human antibody response to natural CCHFV infection presented here provides insights for the development of such a vaccine. The repertoire analysis disclosed herein revealed that the large majority of CCHFV-specific antibodies target the Gc subunit. Most of these antibodies have clean-low polyreactivity, thus showing high specificity to GnGc. Additionally, many of these antibodies are cross-reactive to multiple strains of CCHFV.

Accordingly, disclosed herein are highly selective and potent anti-CCHFV antibodies, as well as possible vaccine candidates, for the treatment and or prophylaxis of CCHFV infection. Additionally, the reagents disclosed here provide a useful set of tools for the evaluation of clinical trials, which will be critical for selecting the optimal CCHFV vaccination or antibody-based therapeutic strategy from those currently under investigation.

In some embodiments, the present disclosure provides an antibody comprising a CCHFV binding domain, CDRL3, wherein the CDRL3 binding domain comprises a consensus motif, the consensus motif comprising the sequence QQX₁X₂X₃X₄X₅X₆X₇, wherein X₁ is S, T, or A, X₂ is F or Y, X₃ is S, T, I, H, or N, X₄ is A or T, X₅ is L or P, X₆ is W, L, S, P, R, I, or Y, and X₇ is T or A. Clone ADI-37836 includes this consensus motif.

In some embodiments, the present disclosure provides an antibody comprising a CCHFV binding domain, CDRL3, wherein the CDRL3 binding domain comprises a consensus motif, the consensus motif comprising the sequence X₁X₂X₃X₄X₅X₆X₇X₈T, wherein X₁ is Q or H, X₂ is Q or H, X₃ is Y or F, X₄ is A, G, S, T, E, or D, X₅ is T, S, or I, X₆ is S or Y, X₇ is P, L, or R, and X₈ is W, F, R, or Y. Clones ADI-36145, ADI-36193 and ADI-42462 include this consensus motif.

In some embodiments, the present disclosure provides an antibody comprising a CCHFV binding domain, CDRL3, wherein the CDRL3 binding domain comprises a consensus motif, the consensus motif comprising the sequence X₁QX₂YX₃X₄X₅X₆T, wherein X₁ is Q or L, X₂ is S, T, or Y, X₃ is S or T, X₄ is N, H, L, I, or V, X₅ is S or P, and X₆ is L or R. Clones ADI-36121, ADI-36122 and ADI-36125 included this consensus motif.

In some embodiments, the present disclosure provides an antibody comprising a CCHFV binding domain, CDRL3, wherein the CDRL3 binding domain comprises a consensus motif, the consensus motif comprising the sequence QQYX₁X₂WPX₃X₄T, wherein X₁ is S or N, X₂ is D or N, X₃ is G, S, P, or T, and X₄ is Y or W. Clone ADI-42623 includes this consensus motif.

In some embodiments, the present disclosure provides an antibody comprising a CCHFV binding domain, CDRL3, wherein the CDRL3 binding domain comprises a consensus motif, the consensus motif comprising the sequence QQX₁X₂X₃WPX₄X₅T, wherein X₁ is F or Y, X₂ is N or G, X₃ is H, N, or K, X₄ is P or L, and X₅ is G, I, or L. Clone ADI-42479 includes this consensus motif.

In some embodiments, the present disclosure provides an antibody comprising a CCHFV binding domain, CDRL3, wherein the CDRL3 binding domain comprises a consensus motif, the consensus motif comprising the sequence QX₁YGX₂SPX₃X₄T, wherein X₁ is H or Q, X₂ is N, T, R, or S, X₃ is E, P, or T, and X₄ is W or Y. Clone ADI-42437 includes this consensus motif.

In some embodiments, the present disclosure provides an antibody comprising a CCHFV binding domain, CDRL2, wherein the CDRL2 binding domain comprises a consensus motif, the consensus motif comprising the sequence X₁X₂SX₃RAX₄, wherein X₁ is D, G, R, A, T, S, or E, X₂ is A, S, T, P, or V, X₃ is N, T, S, R, H, K, or A, and X₄ is T, A, D, or S. Clones ADI-36145; ADI-37847; ADI-42437 ADI-42462 ADI-42479; and ADI-42623 include this consensus motif.

In some embodiments, the present disclosure provides an antibody comprising a CCHFV binding domain, CDRL2, wherein the CDRL2 binding domain comprises a consensus motif, the consensus motif comprising the sequence X₁X₂SX₃LX₄X₅, wherein X₁ is G, T, S, or A, X₂ is A, T, or E, X₃ is Y, S, T, N, E, I, R, or A, X₄is Q, H, E, K, or R, and X₅ is S, R, G, or T. Clone ADI-36121 includes this consensus motif.

In some embodiments, the present disclosure provides an antibody comprising a CCHFV binding domain, CDRL2, wherein the CDRL2 binding domain comprises a consensus motif, the consensus motif comprising the sequence X₁ASX₂LX₃X₄, wherein X₁ is K, D, R, Q, or E, X₂ is N, T, or S, X₃ is E, Q, or K, and X₄ is S, T, N, G, or I. Clone ADI-37801 includes this consensus motif.

In some embodiments, the present disclosure provides an antibody comprising a CCHFV binding domain, CDRL2, wherein the CDRL2 binding domain comprises a consensus motif, the consensus motif comprising the sequence X₁X₂X₃X₄RPS, wherein X₁ is E or D, X₂ is D, V, N, or E, X₃ is Y, N, D, H, or K, X₄ is Q, R, or K. Clone ADI-37849 includes this consensus motif.

In some embodiments, the present disclosure provides an antibody comprising a CCHFV binding domain, CDRH2, wherein the CDRH2 binding domain comprises a consensus motif, the consensus motif comprising the sequence X₁X₂RSEAYX₃GX₄TEYAASVX₅G, wherein X₁ is L or F, X₂ is I, V, or T, X₃ is S, R, or G, X₄ is T or A, and X₅ is R or K. Clone ADI-42462 includes this consensus motif.

In some embodiments, the present disclosure provides an antibody comprising a CCHFV binding domain, CDRL1, wherein the CDRL1 binding domain comprises a consensus motif, the consensus motif comprising the sequence RASQX₁X₂X₃X₄X₅LX₆, wherein X₁ is S, T, A, or N, X₂ is I, V, or L, X₃ is S, G, Y, D, R, or N, X₄ is K, S, T, R, F, G, N or I, X₅ is Y, W, N, S, D, F, or T, and X₆ is S, A, T, or N. Clones ADI-36121; ADI-3-7801; ADI-37847; ADI-42479 and ADI-42623 include this consensus motif.

In some embodiments, the present disclosure provides an antibody comprising a CCHFV binding domain, CDRL1, wherein the CDRL1 binding domain comprises a consensus motif, the consensus motif comprising the sequence X₁ASX₂X₃X₄X₅X₆X₇X₈LA, wherein X₁ is G or R, X₂ is Q or H, X₃ is S or T, X₄ is V, L, or I, X₅ is S, T, Y, or G, X₆ is T, N, S, V, or H, X₇ is N or R, and X₈ is Y or S. Clone ADI-42462 includes this consensus motif.

In some embodiments, the present disclosure provides an antibody comprising a CCHFV binding domain, CDRL1, wherein the CDRL1 binding domain comprises a consensus motif, the consensus motif comprising the sequence RASQX₁X₂X₃X₄X₅X₆X₇X₈, wherein X₁ is S, R, I, or T, X₂ is L, V, or I, X₃ is S, R or T, X₄ is G, S, H, T, or N, X₅ is A, S, or T, X₆ is F, Y, or N, X₇ is V, L, or F, and X₈ is A, T, or V. Clone ADI-42437 includes this consensus motif.

In some embodiments, the present disclosure provides an antibody comprising a CCHFV binding domain, CDRH1, wherein the CDRH1 binding domain comprises a consensus motif, the consensus motif comprising the sequence X₁X₂X₃X₄X₅X₆X₇X₈X₉, wherein X₁ is Y or F, X₂ is T or S, X₃ is F, L, or M, X₄ is T, S, or A, X₅ is S, T, or A, X₆ is F, H, D, N, C, or Y, X₇ is E, S, Y, G, A, W, N, T, or D, X₈ is M, I, L, or V, and X₉ is N, H, S, F, T, I C, G, Y, or L. Clone ADI-42437 includes this consensus motif.

In some embodiments, the present disclosure provides an antibody comprising a CCHFV binding domain, CDRH1, wherein the CDRH1 binding domain comprises a consensus motif, the consensus motif comprising the sequence X₁X₂FX₃X₄X₅X₆X₇Xs, wherein X₁ is F, G, or Y, X₂ is S or T, X₃ is S, T, or R, X₄ is S or T, X₅ is Y, F, H, S, or Q, X₆ is V, R, S, A, or T, X₇ is I or M, and Xs is T or S. Clone ADI-36121 includes this consensus motif.

In some embodiments, the present disclosure provides an antibody comprising a CCHFV binding domain, CDRH1, wherein the CDRH1 binding domain comprises a consensus motif, the consensus motif comprising the sequence X₁X₂FX₃GX₄X₅X₆X₇, wherein X₁ is F or Y, X₂ is S, T, or R, X₃ is S or T, X₄ is Y or S, X₅ is Y, A, T, S, or F, X₆ is M, I, or L, or X₇ is H, Y, N, or I. Clone ADI-37847 includes this consensus motif.

In some embodiments, the present disclosure provides an antibody comprising a CCHFV binding domain, CDRH1, wherein the CDRH1 binding domain comprises a consensus motif, the consensus motif comprising the sequence FTX₁GX₂YX₃X₄X₅, wherein X₁ is L or F, X₂ is E or D, X₃ is A, V, or T, X₄ is L, M, V, or I, and X₅ is S, G, T, or R. Clone ADI-42462 includes this consensus motif.

In some embodiments, the present disclosure provides an antibody comprising a CCHFV binding domain, CDRH1, wherein the CDRH1 binding domain comprises a consensus motif, the consensus motif comprising the sequence X₁SX₂SSX₃X₄X₅X₆WX₇, wherein X₁ is G or D, X₂ is L, V, or I, X₃ is G or S, X₄ is S, D, or G, X₅ is Y or H, X₆ is Y or F, and X₇ is T, A, or S. Clone ADI-37801 includes this consensus motif.

In some embodiments, the present disclosure provides an antibody comprising a CCHFV binding domain, CDRH1, wherein the CDRH1 binding domain comprises a consensus motif, the consensus motif comprising the sequence FX₁FX₂DX₃GMX₄, wherein X₁ is S or T, X₂ is D, A, N, G, X₃ is F or Y, and X₄ is T, S, or H. Clone ADI-36145 includes this consensus motif.

In some embodiments, the present disclosure provides an antibody comprising a CCHFV binding domain, CDRH1, wherein the CDRH1 binding domain comprises a consensus motif, the consensus motif comprising the sequence GX₁IX₂SX₃SX₄X₅WX₆, wherein X₁ is L or S, X₂ is T or S, X₃ is T or S, X₄ is D, L, or Y, X₅ is F or Y, and X₆ is G, A, V, or S. Clones ADI-42479 and ADI-42623 include this consensus motif.

TABLE 1
Germline usage and sequence information of
anti-CCHFV antibodies

| | | VH | LC | | | | | Number |
|---|---|---|---|---|---|---|---|---|
| | | germline | germline | | | | | of nucl |
| Antibody | | gene | gene | CDRH3 | Seq | CDRL3 | Seq | subs in |
| Number | Name | usage | usage | sequence | ID | sequence | ID | VH/VL |
| 1 | ADI- | VH3-48 | VL3-21 | ARDYGLDQ | 1 | QVWDSDSYH | 17 | 16/16 |
| | 36120 | | | | | YV | | |
| 2 | ADI- | VH3-23 | VK1-39 | VKDPKAWLEPEW | 2 | QQSYSNPRT | 18 | 11/7 |
| | 36121 | | | | | | | |
| 3 | ADI- | VH3-7 | VK1-39 | ARDNRAVDGFDI | 3 | QQSYSNLFT | 19 | 10/6 |
| | 36122 | | | | | | | |
| 4 | ADI- | VH3-21 | VK1-27 | ARDHVH | 4 | LYYNSAPWT | 20 | 8/3 |
| | 36125 | | | | | | | |
| 5 | ADI- | VH3-49 | VK3-20 | TRGDYVFVY | 5 | QQYGSSPWT | 21 | 15/6 |
| | 36145 | | | | | | | |
| 6 | ADI- | VH1-18 | VK3-11 | AREQRSTWLNGM | 6 | QHRSTWPPT | 22 | 13/3 |
| | 36193 | | | DV | | | | |
| 7 | ADI- | VH4-31 | VK1-5 | ARDRMDYSGSGV | 7 | QQYGSYSRT | 23 | 12/4 |
| | 37801 | | | FDY | | | | |
| 8 | ADI- | VH1-18 | VL2-8 | TFGEIWANPFDI | 8 | SSYAGSNDI | 24 | 8/0 |
| | 37817 | | | | | AE | | |
| 9 | ADI- | VH4-34 | VK1-39 | ARGTVNFYDNRC | 9 | QQSFSTPRT | 25 | 7/4 |
| | 37836 | | | LDY | | | | |
| 10 | ADI- | VH3-15 | VL9-49 | MTHYGLTY | 10 | GAEHGSGSN | 26 | 8/7 |
| | 37842 | | | | | FLVV | | |
| 11 | ADI- | VH1-2 | VK3-11 | ARDRAEQHFDY | 11 | HLRRNWPPA | 27 | 8/2 |
| | 37847 | | | | | LT | | |
| 12 | ADI- | VH4- | VL2-23 | AREVVEGYYMDV | 12 | CSYVGSSTS | 28 | 7/7 |
| | 37849 | 38-2 | | | | YV | | |
| 13 | ADI- | VH1-18 | VK3-20 | ARVGGSYIGY | 13 | QQYGSSPPY | 29 | 11/2 |
| | 42437 | | | | | T | | |
| 14 | ADI- | VH3-49 | VK3-20 | ARGDYVFVY | 14 | QQYDTSPWT | 30 | 5/8 |
| | 42462 | | | | | | | |
| 15 | ADI- | VH4-39 | VK3-15 | ARPKAVGFYHVG | 15 | QQYNNWPPG | 31 | 12/2 |
| | 42479 | | | YFDL | | T | | |
| 16 | ADI- | VH4-39 | VK3-15 | ARESGYFDY | 16 | QQYNNWPTW | 32 | 4/2 |
| | 42623 | | | | | T | | |

TABLE 2
Affinity and Neutralization data for anti-CCHFV antibodies

| Antibody | | Monovalent GnGc | Neut (35 nM) VLP | Neut (350 nM) VLP |
|---|---|---|---|---|
| Number | Name | Binding (KD) | Avg. % Neutralization | Avg. % Neutralization |
| 1 | ADI-36120 | 1.6E−09 | −10.5 | 45.62 |
| 2 | ADI-36121 | 8.3E−10 | 99.33 | 100.19 |
| 3 | ADI-36122 | 8.9E−10 | NN | 16.71 |
| 4 | ADI-36125 | 1.2E−09 | 64.95 | 91.28 |
| 5 | ADI-36145 | 9.7E−10 | 91.75 | 99.83 |
| 6 | ADI-36193 | 3.9E−09 | 77.95 | 86.69 |
| 7 | ADI-37801 | 2.7E−09 | 90.29 | 90.88 |
| 8 | ADI-37817 | 2.9E−09 | 90.67 | 85.11 |
| 9 | ADI-37836 | 3.1E−09 | 90.42 | 93.97 |
| 10 | ADI-37842 | 3.2E−09 | 90.52 | 84.25 |
| 11 | ADI-37847 | 3.5E−09 | 89.6 | 94.92 |
| 12 | ADI-37849 | 3.6E−09 | 84.81 | 84.41 |
| 13 | ADI-42437 | 2E−09 | 82.16 | 92.43 |
| 14 | ADI-42462 | 1.4E−09 | 20.32 | 52.95 |
| 15 | ADI-42479 | 2.2E−09 | 86.04 | 79.87 |
| 16 | ADI-42623 | 3E−09 | 92.82 | 95.07 |

TABLE 3
Informal Sequence Listing

| Anti- | SEQ | | | |
|---|---|---|---|---|
| body | ID | | Clone # | |
| Number | NO: | Sequence | (ADI) | Descriptors |
| 1 | 33 | QVQLVQSGGGLVRPGGSLRLSCVA | ADI-36120 | Heavy chain |
| | | SGFTFSSFEMNWVRQAPGKGLEWV | | variable region |
| | | AYIGDSGSTIFYADSVQGRFTISR | | (“HC”) amino acid |
| | | DNAKASLYLQMNDLRAEDTAIYYC | | sequence |
| | | ARDYGLDQWGQGTLVTVSS | | |
| 1 | 34 | QPVLTQPPSVSVAPGQTASLTCAG | ADI-36120 | Light chain variable |
| | | HNIGDKSVHWYQQKPGQAPVEVIF | | region (“LC”) |
| | | HDSERPPGIPERFSASNSGNTAIL | | amino acid |
| | | TISRVEAGDEADYHCQVWDSDSYH | | sequence |
| | | YVFGGGTKLTVL | | |
| 2 | 35 | QVQLVQSGGDLVQPGGSLRLSCAA | ADI-36121 | Heavy chain |
| | | SGFTFSSYVMSWVRQAPGKGLEWV | | variable region |
| | | SVIYRGG-STKYADSVKGRFTISR | | (“HC”) amino acid |
| | | DDSKNTLYLQMNSLRVEDTAVYYC | | sequence |
| | | VKDPKAWLEPEWWGQGTLVTVSS | | |
| 2 | 36 | DIVLTQSPSSLSASVGDRVTITCR | ADI-36121 | Light chain variable |
| | | ASQSISKYLSWFQQKPGKAPNLLI | | region (“LC”) |
| | | YAASYLQSGVPSRFSGSGSGTDFT | | amino acid |
| | | LTISSLQPEDFATYYCQQSYSNPR | | sequence |
| | | TFGQGTKVEIK | | |
| 3 | 37 | QVQLVQSGGGLVRPGGSLRLSCAA | ADI-36122 | Heavy chain |
| | | SGFTFTTFRMSWVRQAPGKGLEWV | | variable region |
| | | ANINQDSSEKYYVDSVKGRFSISR | | (“HC”) amino acid |
| | | DNAKNSLYLQMNSLRAEDTAVYYC | | sequence |
| | | ARDNRAVDGFDIWGQGTTVTVSS | | |
| 3 | 38 | ETTLTQSPSSLSASVGDRVTITCR | ADI-36122 | Light chain variable |
| | | ASQSIYSYLNWYQKKIGKAPKLLI | | region (“LC”) |
| | | YAASSLQSGVPSRFSGSGSGTDFT | | amino acid |
| | | LTISSLQPEDIATYYCQQSYSNLF | | sequence |
| | | TFGPGTKVEIK | | |
| 4 | 39 | QVQLVQSGGGLVKPGGSLRLSCAA | ADI-36125 | Heavy chain |
| | | SGFSLSSYSMNWVRQAPGKGLEWV | | variable region |
| | | SSISNTGSYKYYADSVKGRFTISR | | (“HC”) amino acid |
| | | DNAKNSVYLQMNSLRAEDRAVYYC | | sequence |
| | | ARDHVHWGQGTLVTVSS | | |
| 4 | 40 | ETTLTQSPSSLSASVGDRVTITCR | ADI-36125 | Light chain variable |
| | | ASQGISNFLAWYQQKPGKVPKLLI | | region (“LC”) |
| | | YTTSTLQSGVPSRFSGSGSGTDFT | | amino acid |
| | | LTISS | | sequence |
| 5 | 41 | EVQLVESGGGLVQPGRSLRLSCSA | ADI-36145 | Heavy chain |
| | | SGFTFGDYGMTWVRQAPGKGLEWV | | variable region |
| | | GLVRSEAYRGTTEYAASVRGRFTI | | (“HC”) amino acid |
| | | SRDNARNIAYLHMNSLKTEDTGVY | | sequence |
| | | YCTRGDYVFVYWGQGTLVTVSS | | |
| 5 | 42 | ETTLTQSPGTLSLSPGERATLSCR | ADI-36145 | Light chain variable |
| | | ASQSVSNNYLAWYQQKPGQAPRFL | | region (“LC”) |
| | | IYRASSRATGIPDRFSGTGSGTDF | | amino acid |
| | | TLTISRLEPEDFAVYYCQQYGSSP | | sequence |
| | | WTFGQGTKVDIK | | |
| 6 | 43 | QITLKESGAEVKKPGASVKVSCKA | ADI-36193 | Heavy chain |
| | | SGYTIGSYGISWVRQAPGQGLEWM | | variable region |
| | | GWISGNNDNTNYVEKFQGRVIMTI | | (“HC”) amino acid |
| | | DTSTSTAYMELRSLTSDDTAVYYC | | sequence |
| | | AREQRSTWLNGMDVWGQGTTVTVS | | |
| | | S | | |
| 6 | 44 | EIVMTQSPATLSLSPGERATLSCR | ADI-36193 | Light chain variable |
| | | ASQSVSRYLAWYQQKPGQAPRLLI | | region (“LC”) |
| | | YDSSNRATGVPARFSGSGSGTDFT | | amino acid |
| | | LTISSLEPEDFAVYYCQHRSTWPP | | sequence |
| | | TFGPGTKVEIK | | |
| 7 | 45 | QVQLVESGPGLLKPSQTLSLTCTV | ADI-37801 | Heavy chain |
| | | SGGSLSSGGYYWSWIRQHPGQGLE | | variable region |
| | | CIGYIYYSGSTYYSPSLESRVDIS | | (“HC”) amino acid |
| | | MDTSMNQFSLKLRSVTAADTAVYY | | sequence |
| | | CARDRMDYSGSGVFDYWGQGTLVT | | |
| | | VSS | | |
| 7 | 46 | EIVLTQSPSTLSASVGDRVTITCR | ADI-37801 | Light chain variable |
| | | ASQSISRWLAWYQQKPGKAPRLLI | | region (“LC”) |
| | | HKASSLESGVPSRFSGSGSGTEFT | | amino acid |
| | | LTITSLQPDDFATYYCQQYGSYSR | | sequence |
| | | TFGQGTKVEIK | | |
| 8 | 47 | QVQLVQSGAEVKKPGASVNVSCKA | ADI-37817 | Heavy chain |
| | | SGYTFPSYGISWVRQAPGQGLEWM | | variable region |
| | | GWISPYSGNTNYAQKLQGRVIMTT | | (“HC”) amino acid |
| | | DPSTSTAYMDLRSLTSDDTAVYYC | | sequence |
| | | TFGIHWANPFDIWSQGTTVTVSS | | |
| 8 | 48 | QPVLTQPPSASGSPGQSVTISCTG | ADI-37817 | Light chain variable |
| | | TSSDVGGYNYVSWYQQHPGKAPKL | | region (“LC”) |
| | | MIYEVSKRPSGVPDRFSGSKSGNT | | amino acid |
| | | ASLTVSGLQAEDEADYYCSSYAGS | | sequence |
| | | NDFEFGGGTKLTVL | | |
| 9 | 49 | QVQLQQWGAGLLKPSETLSLTCAV | ADI-37836 | Heavy chain |
| | | YGGSFSGYYWSWIRQSPGKGLEWI | | variable region |
| | | GEINHSGTTHYNPSLNSRVTMSVD | | (“HC”) amino acid |
| | | TSKSQFSLNLSSVTAADTAVYYCA | | sequence |
| | | RGTVNFYDNRCLDYWGQGTLVTVS | | |
| | | S | | |
| 9 | 50 | DIQMTQSPSSLSASVGDRVIITCR | ADI-37836 | Light chain variable |
| | | ASHSISSYLNWYQQKAGKAPKLLI | | region (“LC”) |
| | | YAASSLQSGVPSRFSGSGSGTDFS | | amino acid |
| | | LTISSLQPEDFATYYCQQSFSTPR | | sequence |
| | | TFGQGTKVDIK | | |
| 10 | 51 | EVQLVESGGGLVKPGGSLRLSCAA | ADI-37842 | Heavy chain |
| | | SGFTFSHGWMSWVRQAPGKGLEWV | | variable region |
| | | GRIKRKTDAGTIDYAAAVKGRFTI | | (“HC”) amino acid |
| | | SRDDSKNTLYLQMNSLKMEDTAVY | | sequence |
| | | YCMTHYGLTYWGQGTLVTVSS | | |
| 10 | 52 | QPVLTQPPSASASLGASVTLTCTL | ADI-37842 | Light chain variable |
| | | SSDYSTYKVDWYQQRPAKGPRFVM | | region (“LC”) |
| | | RVGTGGIVGSKGDGIPDRFSALGS | | amino acid |
| | | GLNRYLIIKNIQQEDEGDYHCGAE | | sequence |
| | | HGSGSNFLVVFGGGTKLTVL | | |
| 11 | 53 | QVQLVQSGAEVKKPGASVKVSCKA | ADI-37847 | Heavy chain |
| | | SGYSFTGYSMHWVRQAPGQGLEWM | | variable region |
| | | GWISPSSGVANYAQKFQGRVTMTT | | (“HC”) amino acid |
| | | DTSITTAYMELSRLRSDDTAVYYC | | sequence |
| | | ARDRAEQHFDYWGQGTLVTVSS | | |
| 11 | 54 | EIVMTQSPATLSLSPGERATLSCR | ADI-37847 | Light chain variable |
| | | ASQSLSSYLAWYQQKPGQAPRLLI | | region (“LC”) |
| | | YDTSNRATGIPARFSGSGSGTDFT | | amino acid |
| | | LTISSLEPEDFAVYYCHLRRNWPP | | sequence |
| | | ALTFGGGTKVEIK | | |
| 12 | 55 | QVQLVESGPGLVKPSETLSLTCAV | ADI-37849 | Heavy chain |
| | | SGYSISIGYFWGWIRQPPGKGLEW | | variable region |
| | | IGSIYHGGSTYYNPSLKSRVTMSV | | (“HC”) amino acid |
| | | DTSKNQFSLKLRSVTAADTAIYYC | | sequence |
| | | AREVVEGYYMDVWGKGTTVTVSS | | |
| 12 | 56 | QSALTQPASVSGSPGQSITISCTG | ADI-37849 | Light chain variable |
| | | TSSHVGNYNLVSWYQHHPGKAPKL | | region (“LC”) |
| | | VISEVNKRPSGISNRFSGSKSGNT | | amino acid |
| | | ASLTISGLQAEDEADYYCCSYVGS | | sequence |
| | | STSYVFGGGTKLTVL | | |
| 13 | 57 | QVQLVQSGPEVKKPGASVKVSCKA | ADI-42437 | Heavy chain |
| | | SGYTFSTYGIIWVRQAPGQGPECI | | variable region |
| | | GWISAHNGNTKYAQNLQGRLTLTT | | (“HC”) amino acid |
| | | DTSTSTAYMELRSLRSDDTAVYYC | | sequence |
| | | ARVGGSYIGYWGQGTLVTVSS | | |
| 13 | 58 | EIVLTQSPGTLSLSPGERATLSCR | ADI-42437 | Light chain variable |
| | | ASQSVTNSYLAWYQQKPGQAPRLL | | region (“LC”) |
| | | IYGASSRATGIPDRFSGSGSGTDF | | amino acid |
| | | TLTISRLEPEDFAVYYCQQYGSSP | | sequence |
| | | PYTFGQGTKVEIK | | |
| 14 | 59 | EVQLVESGGGLVQPGRSLRLSCTA | ADI-42462 | Heavy chain |
| | | SGFTFGDYAMSWVRQAPGKGLEWV | | variable region |
| | | GFIRSEAYGGATEYAASVKGRFTI | | (“HC”) amino acid |
| | | SRDNSKSIAYLQMNSLRTEDTALY | | sequence |
| | | YCARGDYVFVYWGQGALVTVSS | | |
| 14 | 60 | EIVMTQSPGTLSLSLGERATLSCR | ADI-42462 | Light chain variable |
| | | ASQSVSSNYLAWYQQKPGQAPELL | | region (“LC”) |
| | | IYRASSRATGIPDRFSGSGSGTDF | | amino acid |
| | | TLTISTLEPEDFAVYYCQQYDTSP | | sequence |
| | | WTFGQGTKVEIK | | |
| 15 | 61 | QVQLVESGPGLVKPSETLSLTCTV | ADI-42479 | Heavy chain |
| | | SGGSISSSSLFWGWIRQSPGKGPE | | variable region |
| | | WIGSIYDSVNTYYNPSLQSRVTIS | | (“HC”) amino acid |
| | | VDTSKNQFSLNLRSVTVADTAVYY | | sequence |
| | | CARPKAVGFYHVGYFDLWGRGTTV | | |
| | | TVSS | | |
| 15 | 62 | DIVMTQTPATLSVSPGERATLSCR | ADI-42479 | Light chain variable |
| | | ASQSVSSSLAWYQQKTGQAPRLLI | | region (“LC”) |
| | | YGASTRATGIPARFSGSGSGTEFT | | amino acid |
| | | LTISSLQSEDFAVYYCQQYNNWPP | | sequence |
| | | GTFGQGTKVEIK | | |
| 16 | 63 | EVQLLESGPGLVKPSETLSLTCTV | ADI-42623 | Heavy chain |
| | | SGGLISSSSYYWGWIRQPPGKGLE | | variable region |
| | | WIGSISYSGRTYYNPSLKSRVTIS | | (“HC”) amino acid |
| | | VDTSKNQFSLKLSSVTAADTAVYY | | sequence |
| | | CARESGYFDYWGQGTLVTVSS | | |
| 16 | 64 | DIQMTQSPATLSVSPGERATLSCR | ADI-42623 | Light chain variable |
| | | ASQSVGSYLAWYQQKPGQAPRLLI | | region (“LC”) |
| | | YGASTRATGIPARFSGSGSGTEFT | | amino acid |
| | | LTISSLQSEDFAVYYCQQYNNWPT | | sequence |
| | | WTFGQGTKVEIK | | |

### Materials and Methods

### Study Design

To profile the antibody response to CCHFV, peripheral blood mononuclear cells were obtained from adult CCHFV-convalescent donors, and monoclonal antibodies from CCHFV-reactive B cells were isolated therefrom. The antibodies were characterized by sequencing, binding, epitope mapping, and neutralization assays. All samples for this study were collected with informed consent of volunteers. This study was unblinded and not randomized. At least two independent experiments were performed for each assay.

### Generation of CCHFV Sorting Probes

The CCHFV GnGc and GP38 glycoproteins were engineered (transmembrane regions deleted) to be secreted as soluble recombinantly expressed proteins from Schneider 2 insect cells. The proteins were then purified using standard Strep II tag purification techiniques. The purified proteins were then complexed with labled streptavidin probes (utilizing the Strep-II tags fused to the proteins) together providing the antigen specific probes required to support single cell fluorescence activated B-cell sorting and resulting antibody isolation from CCHFV-convalescent adult human donors (see FIG. 1A).

### Single B-Cell Sorting

Peripheral blood mononuclear cells from previously CCHF-infected convalescent donors were stained using anti-human CD19 (PE-Cy7), CD20 (PE-Cy7), CD3 (PerCP-Cy5.5), CD8 (PerCP-Cy5.5), CD14 (PerCP-Cy5.5), CD16 (PerCP-Cy5.5), IgM (AF-488), IgD (BV421), CD27 (BV510), PI, and a mixture of dual-labeled IbAr10200 GnGc tetramers (25 nM each). Tetramers were prepared fresh for each experiment, and total B cells binding to the GnGc tetramers were single cell sorted. Single cells were sorted using a BD FACS Aria II (BD Biosciences) into 96-well PCR plates (BioRAD) containing 20 μL/well of lysis buffer [5 μL of 5× first strand cDNA buffer (Invitrogen), 0.625 μL of NP-40 (New England Biolabs), 0.25 μL RNaseOUT (Invitrogen), 1.25 μL dithiothreitol (Invitrogen), and 12.6 μL dH2O]. Plates were immediately stored at −80° C.

### Amplification and Cloning of Antibody Variable Genes

Antibody variable genes (IgH, IgK, and IgL) were amplified by reverse transcription PCR and nested PCRs using cocktails of IgG-, IgA-, and IgM-specific primers, as described previously (Tiller et al, J Immunol 2008). The primers used in the second round of PCR contained 40 base pairs of 5′ and 3′ homology to the digested expression vectors, which allowed for cloning by homologous recombination into S. cerevisiae. The lithium acetate method for chemical transformation was used to clone the PCR products into S. cerevisiae (Gietz and Schiestl, Nat Protoc 2007). 10 uL of unpurified heavy chain and light chain PCR product and 200 ng of the digested expression vectors were used per transformation reaction. Following transformation, individual yeast colonies were picked for sequencing and characterization.

### Expression and Purification of IgGs and Fab Fragments

IgGs were expressed in S. cerevisiae cultures grown in 24-well plates, as described previously (Bornholdt et al, Science 2016b). After 6 days, the cultures were harvested by centrifugation and IgGs were purified by protein A-affinity chromatography. The bound antibodies were eluted with 200 mM acetic acid/50 mM NaCl (pH 3.5) into ⅛th volume 2 M Hepes (pH 8.0), and buffer-exchanged into PBS (pH 7.0).

### Biolayer Interferometry Binding Analysis

IgG binding to GnGc (IbAr10200), GnGc (China), GnGc (Kosovo), and Gc (IbAr10200) was measured by biolayer interferometry (BLI) using a FortéBio Octet HTX instrument (Pall Life Sciences). For high-throughput KD determination, IgGs were immobilized on anti-human IgG quantitation biosensors (Pall Life Sciences) and exposed to 100nM antigen in PBS with 0.1% BSA (PBSF) for an association step, followed by a dissociation step in PBSF. Data were analyzed using the ForteBio Data Analysis Software 7. Kd values were calculated for antibodies with BLI responses >0.1 nm, and the data were fit to a 1:1 binding model to calculate association and dissociation rate constants. The KD values were calculated using the ratio kd/ka.

### Polyreactivity Assay

Polyspecificity reagent binding was assessed as previously described (Xu et al, Protein Eng Des Sel 2013). Briefly, soluble membrane protein (SMP) and soluble cytosolic protein (SCP) fractions were prepared from Chinese hamster ovary cells and biotinylated with NHS-LC-Biotin reagent (Pierce, ThermoFisher Cat#21336). 2 million IgG-presenting yeast were transferred to a 96-well assay plate, pelleted to remove supernatant, then the pellets were resuspended in 50 uL of 1:10 diluted stock of biotinylated SCPs and SMPs and incubated on ice for 20 minutes. Cells were washed twice with ice-cold PBSF, and the samples were incubated in 50 uL of secondary labeling mix (Extravadin-R-PE, goat F(ab') 2-anti human kappa-FITC, and propidium iodide) on ice for 20 minutes. The samples were analyzed for polyspecificity reagent binding using a FACSCanto II (BD Biosciences) with HTS sample injector. Flow cytometry data were analyzed for mean fluorescence intensity in the R-PE channel and normalized to three control antibodies exhibiting low, medium, and high MFI values.

### Antibody Epitope Binning

Biotinylated CCHFV Gn/Gc IbAr10200 (15 nM or 50 nM) was incubated with a twentyfold excess of Fab (300 nM or 1 uM respectively) for 30 min at room temperature before mixing with yeast expressing monoclonal anti-CCHFV Gn/Gc IgG. After washing with PBSF to remove unbound antigen, the bound antigen was detected using streptavidin Alexa Fluor 633 (Life Technologies) at a 1:500 dilution and antibody light chain was detected using Goat F(ab′)2 anti-human kappa FITC and Goat F(ab′)2 and anti-human lambda FITC (SouthernBiotech) both at a 1:100 dilution. The samples were analyzed by flow cytometry using FACSCanto II. The amount of antigen bound was normalized to the light chain FITC level. Competition level was determined by the fold reduction in antigen binding in the presence of a competitor Fab compared to antigen binding in the absence of competition. Antibodies with greater than tenfold reduction were considered to be in competition with the precomplexed Fab.

### CCHFV Virus-Like Particle Production and Purification

Virus-like particles (VLPs) carrying CCHFV Gn/Gc were produced as previously described (Zivcec M et al. 2015. PLoS Negl Trop Dis 9(12): e0004259). Briefly, BSR-T7 cells were transfected with plasmids encoding NP, GPC, L, T7-polymerase, and a Luciferase-expressing minigenome. 3 days post-transfection, supernatant was collected and VLPs pelleted through ultracentrifugation. Pelleted VLPs were resuspended in complete DMEM (2% FBS, 1% Penicillin-streptomycin, 1% GlutaMax) (Life Technologies, Grand Island, N.Y.) prior to storage at −80° C.

### Screening of mAb Neutralization Potential Using CCHFV VLPs

Gn/Gc specific mAbs were diluted to 350 and 35 nM in complete DMEM. Pelleted VLP dilutions in complete DMEM were determined empirically in order to reach a level at which maximal Luciferase signal is >100-fold higher than background. Diluted VLPs were added to the mAbs and the mixture was incubated at 4° C. for 1 hour. Confluent Vero African grivet kidney cells were infected with the VLP: mAb mixture, followed by incubation at 37° C. for ˜16 hours. The inoculum was then aspirated, and cells washed with phosphate buffered saline. NanoLuciferase substrate (Promega, Madison, Wis.) was added to infected cells and following a 5-minute incubation luminescence was measured using 10 second integration in a Perkin Elmer Victor 2 microplate reader. Based on two technical replicates per mAb, neutralizing potential was calculated as a ratio of signal compared to VLP signal in the absence of mAb.

### Analysis of Synergistic Neutralization

Neutralizing mAbs were analyzed for synergy by performing neutralization assays of 1:1 molar ratio mixtures of parental antibodies and comparing the neutralization potential to the parental mAbs alone. Briefly, dilution series of neutralizing mAbs were made in complete DMEM. VLPs were diluted as above and then added to the mAbs, followed by incubation at 4° C. for 1 hour. Infection and luminescence readings were performed as above. Resulting mAb neutralization curves were analyzed for synergy using combination index analysis (Chou T-C and Talalay P, 1984 Adv Biol Regul (22). pp 27-55). Combination index values <1 at relevant effect sizes were considered indicative of synergy.

What is claimed is:

1. An isolated human antibody or an antigen-binding fragment thereof that specifically binds to a Crimean Congo Hemorrhagic Fever Virus (CCHFV) protein, wherein at least one of the CDRH1, a CDRH2, a CDRH3, a CDRL1, a CDRL2, and CDRL3 amino acid sequence of the antibody or the antigen-binding fragment thereof is at least 70% identical to at least one the CDRH1, a CDRH2, a CDRH3, a CDRL1, a CDRL2, and/or a CDRL3 amino acid sequences disclosed in Table 3 of an antibody selected from Antibody Number 1 through Antibody Number 16 as disclosed in Table 3; and wherein said antibody or the antigen-binding fragment thereof also has one or more of the following characteristics:
a) the antibody or antigen-binding fragment thereof cross-competes with said antibody or antigen-binding fragment thereof for binding to CCHFV;
b) the antibody or antigen-binding fragment thereof displays a clean or low polyreactivity profile;
c) the antibody or antigen-binding fragment thereof displays neutralization activity toward CCHFV in vitro;
d) the antibody or antigen-binding fragment thereof displays an in vitro neutralization potency (IC₅₀) of between about 0.5 microgram/milliliter (μg/ml) to about 5 μg/ml; or
e) the antibody or antigen-binding fragment thereof binds to at least one of Gn, Gc, and a GnGc complex.

2. The isolated antibody or antigen-binding fragment thereof of claim 1, wherein the antibody or antigen-binding fragment thereof comprises: at least two of characteristics a) through e).

3. The isolated antibody or antigen-binding fragment thereof of claim 2, wherein the antibody or antigen-binding fragment thereof comprises at least one of:
a) the CDRH3 amino acid sequence of any one of the antibodies designated Antibody Number 1 through Antibody Number 16 as disclosed in Table 3;
b) the CDRH2 amino acid sequence of any one of the antibodies designated Antibody Number 1 through Antibody Number 16 as disclosed in Table 3;
c) the CDRH1 amino acid sequence of any one of the antibodies designated Antibody Number 1 through Antibody Number 16 as disclosed in Table 3;
d) the CDRL3 amino acid sequence of any one of the antibodies designated Antibody Number 1 through Antibody Number 16 as disclosed in Table 3;
e) the CDRL2 amino acid sequence of any one of the antibodies designated Antibody Number 1 through Antibody Number 16 as disclosed in Table 3; or
f) the CDRL1 amino acid sequence of any one of the antibodies designated Antibody Number 1 through Antibody Number 16 as disclosed in Table 3.

4. The isolated antibody or antigen-binding fragment thereof of claim 1, wherein the antibody or antigen-binding fragment thereof comprises:
a) a heavy chain (HC) amino acid sequence of any one of the antibodies designated Antibody Number 1 through Antibody Number 16 as disclosed in Table 3; and
b) a light chain (LC) amino acid sequence of any one of the antibodies designated Antibody Number 1 through Antibody Number 16 as disclosed in Table 3.

5. The isolated antibody or antigen-binding fragment thereof of claim 1 , wherein the antibody is selected from the group consisting of antibodies that are at least 80% identical to any one of the antibodies designated as Antibody Number 1 through Antibody Number 16 as disclosed in Table 3.

6. The isolated antibody or antigen-binding fragment thereof of claim 4, wherein the antibody is selected from the group consisting of the antibodies designated as Antibody Number 1 through Antibody Number 16 as disclosed in Table 3.

7. An isolated nucleic acid sequence encoding an antibody or antigen-binding fragment thereof according to claim 1.

8. An expression vector comprising the isolated nucleic acid sequence according to claim

9. A host cell transfected with the expression vector according to claim 8.

10. A pharmaceutical composition comprising: one or more of the isolated antibodies or antigen-binding fragments thereof according to claim 1 and a pharmaceutically acceptable carrier and/or excipient.

11. A method of treating or preventing a Crimean Congo Hemorrhagic Fever Virus (CCHFV) infection comprising administering to a patient in need thereof one or more antibodies or antigen-binding fragments thereof according to claim 1.

12. The method according to claim 11, wherein the method further comprises administering to the patient a second therapeutic agent.

13. The method according to claim 12, wherein the second therapeutic agent is selected group consisting of: an antiviral agent, a vaccine specific for CCHFV, an siRNA specific for an CCHFV antigen or a second antibody specific for a CCHFV antigen.

14. A CCHFV antibody and/or antigen-binding fragment comprising a CCHFV binding domain, CDRL3, wherein the CDRL3 binding domain comprises a consensus motif comprising the sequence:
a) X₁X₂X₃X₄X₅X₆X₇X₈T, wherein X₁ is Q or H, X₂ is Q or H, X₃ is Y or F, X₄ is A, G, S, T, E, or D, X₅ is T, S, or I, X₆ is S or Y, X₇ is P, L, or R, and X₈ is W, F, R, or Y;
b) X₁QX₂YX₃X₄X₅X₆T, wherein X₁ is Q or L, X₂ is S, T, or Y, X₃ is S or T, X₄ is N, H, L, I, or V, X₅ is S or P, and X₆ is L or R;
c) QQYXiX₂WPX₃X₄T, wherein X₁ is S or N, X₂ is D or N, X₃ is G, S, P, or T, and X₄ is Y or W;
d) QQX₁X₂X₃WPX₄X₅T, wherein X₁ is F or Y, X₂ is N or G, X₃ is H, N, or K, X₄ is P or L, and X₅ is G, I, or L; or
e) QX₁YGX₂SPX₃X₄T, wherein X₁ is H or Q, X₂ is N, T, R, or S, X₃ is E, P, or T, and X₄ is W or Y.
